(19) United States
(12) Patent Application Publication
(10) Pub. No.: US 2019/0351068 A1
(43) Pub. Date: Nov. 21, 2019

(54) COMPOSITIONS AND METHODS FOR DRUG DELIVERY

(71) Applicant: Shen Pang, Santa Monica, CA (US)

(72) Inventor: Shen Pang, Santa Monica, CA (US)

(21) Appl. No.: 16/204,739

(22) Filed: Nov. 29, 2018

Related U.S. Application Data
(60) Provisional application No. 62/592,057, filed on Nov. 29, 2017.

(51) Int. Cl. A61K 47/69; A61K 47/18; A61K 47/62; A61K 9/51

(52) U.S. Cl. CPC A61K 47/6931; A61K 47/18; B82Y 5/00; A61K 9/5146; A61K 47/62

(57) ABSTRACT

Provided herein is are polymeric nanoparticles and polymer-bioactive agent conjugates capable of delivering therapeutic agents to the central nervous system (CNS). Further provided herein is a method of treating diseases with such polymer nanoparticles and polymer-bioactive agents conjugates. Also provided herein is a method of making the polymeric nanoparticles.

### RELATED APPLICATIONS

This application claims the benefit of U.S. Provisional Application No. 62/592,057, filed Nov. 29, 2017, the contents of which are fully incorporated by reference herein.

### BACKGROUND

Central nervous system (CNS) related diseases and disorders have become are a major cause of mortality globally. The blood-brain barrier (BBB) is a highly restrictive barrier that separates the circulating blood from the CNS, and it is this barrier that has inhibited the development of efficient CNS treatments. (Banks, W. A., Nat. Rev. Drug. Discov. 15, 275-292 (2016); Abbott, N. J., Ronnback, L. & Hansson, E., Nat Rev Neurosci. 7, 41-53 (2006)). Whilst some small molecules can pass through the BBB through the paracellular aqueous and transcellular lipophilic pathways or via transport proteins; the transport of biomacromolecules across the BBB generally relies on receptor mediated or adaptive transcytosis. The receptor mediated transcytosis pathway is generally limited to specific endogenous molecules; whilst the adsorptive pathway results in poor transport efficiency.

To date, various approaches have been explored to enhance the penetration of BBB, which include, but, are not limited to: (1) improving the circulation time of therapeutic agents, (2) chemically modifying therapeutic agents to enable their adsorptive transcytosis, (3) conjugating a peptide or peptidomimetic monoclonal antibody (mAb) to induce a receptor-mediated transcytosis, (4) utilizing “Trojan Horse” carriers to deliver therapeutic antibodies, (5) delivering therapeutic agents through trafficking immune cells, and (6) utilizing physical methods such as ultrasonic wave to open the tight junctions of BBB. However, the efficiency of these methods remains low.

Accordingly, there is an unmet need for a delivery platform that enables effective delivery of therapeutic agents to the CNS for the treatment of CNS-related diseases and disorders.

### SUMMARY OF THE INVENTION

In one aspect, this invention provides polymeric nanoparticles comprising a cross-linked polymer matrix, a plurality of bioactive agents encapsulated in the matrix, and a plurality of transport moieties coupled to the polymer, wherein the transport moieties enhance penetration of the nanoparticle across a blood brain barrier. In certain embodiments, the copolymer comprises agent monomers, each comprising an agent-association moiety that associates (non-covalently) with the agent.

In another aspect, this invention provides polymeric nanoparticles comprising a cross-linked polymer, a plurality of bioactive agents covalently coupled to the polymer, and a plurality of transport moieties coupled to the polymer, wherein the transport moieties enhance penetration of the nanoparticle across a blood brain barrier.

In yet another aspect, this invention provides polymer-bioactive agent conjugates comprising a bioactive agent, a plurality of polymers covalently coupled to the bioactive agent, and a plurality of transport moieties coupled to the polymers, wherein the transport moieties enhance penetration of the nanoparticle across a blood brain barrier.

### BRIEF DESCRIPTION OF THE DRAWINGS

FIG. 1. Molecular structures of acetylcholine, choline and 2-methacryloyloxyethyl phosphorylcholine and quaternized dimethylaminoethyl methacrylate, and schematic illustration of the potential binding mechanism with acetylcholine receptor. Circles indicate structural similarity.

FIG. 2A. Schematic illustration of the synthesis of BBB-penetrative protein nanoparticles. In situ polymerization of monomers and cross-linkers forms a nanoparticle encapsulating the protein molecule; the MPC moieties within the nanoparticle allow effective BBB penetration of the nanoparticles. Upon degradation of the cross-linkers in the nanoparticle, the therapeutic protein can be released within the CNS.

FIG. 2B. Schematic illustration of the synthesis of BBB-penetrative therapeutics by conjugating polymer chains with analogous structures for ChT or nAchRs onto a therapeutic molecule such as protein.

FIG. 3A. Representative TEM image of the HRP nanoparticles.

FIG. 3B. Representative TEM image of the CSF of rhesus macaque monkey. The CSF sample was collected at the first day post intravenous injection with 10.0 mg/kg n-HRP nanoparticles.

FIG. 3C. Concentration of n-HRP in the CSF of rhesus macaque monkey recorded at different doses. The result suggests that n-HRP delivery proceeded in a dose-dependent manner.

FIG. 3D. Pharmacokinetics of n-HRP in the plasma of rhesus macaque monkey at different doses (2.5, 5, and 10 mg/mL).

FIG. 3E. Delivery efficiency of n-HRP, in which the concentration in the CSF was up to ˜5.3% of the plasma concentration.

FIG. 3F. Penetration capability of n-B SA prepared with various monomers crossing the BBB transwell model in vitro. QDMAEMA indicates quaternized dimethylaminoethyl methacrylate.

FIG. 4A. Concentration of Rituximab in the plasma of rhesus macaque monkey via intravenous injection of native Rituximab and Rituximab nanoparticles (n-Rituximab).

FIG. 4B. Concentration of Rituximab in the CSF of rhesus macaque monkey via intravenous injection of native Rituximab and Rituximab nanoparticles (n-Rituximab).

FIG. 5A. Bioluminescence imaging (BLI) of nude mice bearing orthotopic glioma in different treatment groups. Images were recorded at day 10, 20, 30, 40, and 50 after tumor implantation. The mice given the native Nimotuzumab had a similar tumor size to the non-treated group, whereas the group given n(Nimotuzumab) showed a significantly decreased tumor size.

FIG. 5B. H&E staining of the brain sections of nude mice bearing orthotopic glioma in different treatment groups.

FIG. 5C. BLI quantification of the size of orthotopic glioma in nude mice in different treatment groups.

FIG. 5D. Survival rates of the mice given different treatments. The Kaplan-Meier analysis is used. *p<0.05 FIG. 6A. Optical imaging of mouse brain 1 day after intravenous injection with PBS, native BSA (C-nBSA-1), and BSA conjugates (C-nBSA-2).

FIG. 6B. Quantified fluorescence intensity of mouse brain 1 day after intravenous injection with PBS, native BSA (C-nBSA-1), and BSA conjugates (C-nBSA-2).

FIG. 7A. Bioluminescence imaging of the orthotopic U87-EGFRwt glioma xenograft mice and ex vivo fluorescence images of the brains 4 h after one-time injection of 5 mg/kg Cy5.5-labeled n(Nimo) made with different MPC contents.

FIG. 7B. Relative fluorescence intensity of the glioma-bearing brain tissue 4 h after one-time injection of 5 mg/kg Cy5.5-labeled n(Nimo) made with different MPC contents. *p<0.05 and ***p<0.001 (Ordinary one-way ANOVA).

FIG. 8A. Bioluminescence imaging of the orthotopic U87-EGFRwt glioma xenograft mice and ex vivo fluorescence images of the dissected brain 4 hours after one-time injection of 5 mg/kg Cy5.5-labeled n(Nimo). The mice were intraperitoneally pre-injected with different doses of hemicholinium-3 (HC-3) 20 mins prior to the injection of n(Nimo).

FIG. 8B. Relative fluorescence intensity of the glioma-bearing brain tissue 4 h after one-time injection of 5 mg/kg Cy5.5-labeled n(Nimo). The mice were intraperitoneally pre-injected with different doses of hemicholinium-3 (HC-3) 20 mins prior to the injection of n(Nimo). *p<0.05 and ***p<0.001 (Ordinary one-way ANOVA).

FIG. 9A. Bioluminescence imaging of the ischemic (transient focal cerebral ischemia) model rats (upper) and ex vivo fluorescence images of the collected brain tissues (lower) 24 hours after intravenous administration of the Cy5.5 labeled miR-21 nanocapsule (n(Cy5.5-miR-21)) at a dosage of 0.5 mg/kg miR-21, and equal volume PBS was administrated as control.

FIG. 9B. ROI analysis of fluorescent signals and mature miR-21 levels of the collected brain tissues. Data are presented as mean±SD (n=5). *P<0.05 and **P<0.001 (Turkey's post-test following ordinary two-way ANOVA).

FIG. 10A. Representative fluorescence images of the ischemic hemisphere of brain tissues 24 h after intravenous (i.v.) administration of the FITC labeled miR-21 nanocapsule (n(FITC-miR-21)) at a dosage of 0.5 mg/kg miR-21. Equal volume PBS was administrated as control. Green color represents FITC-labeled miR-21. Brain sections were counter-stained with Hoechst for nuclei, and Neuron specific enolase (NSE) for neurons. Scale bar: 100 m.

FIG. 10B. Quantification of FITC intensity and percentages of FITC positive neurons from images.

FIG. 11. Neurologic function of transient focal cerebral ischemia model rats after samples administration. The arrows indicate the sample administration time point. Neurologic deficit score was tested every other day before each injection. Data are presented as mean±SEM. (n=10). *P<0.05 and **P<0.01 (Bonferroni's multiple comparisons test following two-way RM ANOVA).

FIG. 12. TTC staining of brain tissues obtained from the sacrificed rats at day 7.

FIG. 13. Quantified cerebral infarct volume. Data are presented as mean±SEM (n=5). *P<0.05 and **P<0.01. (Turkey's post-test following ordinary one-way ANOVA).

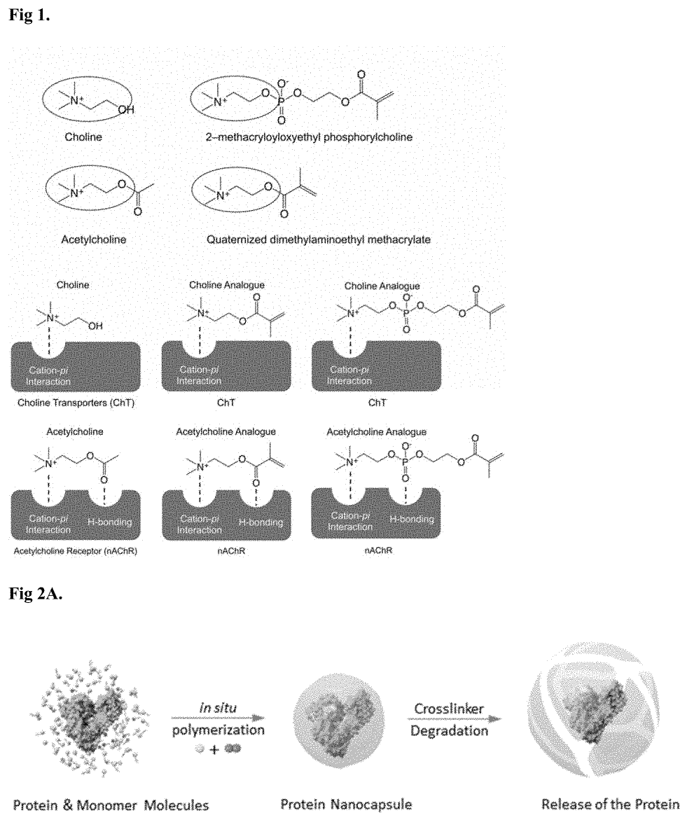

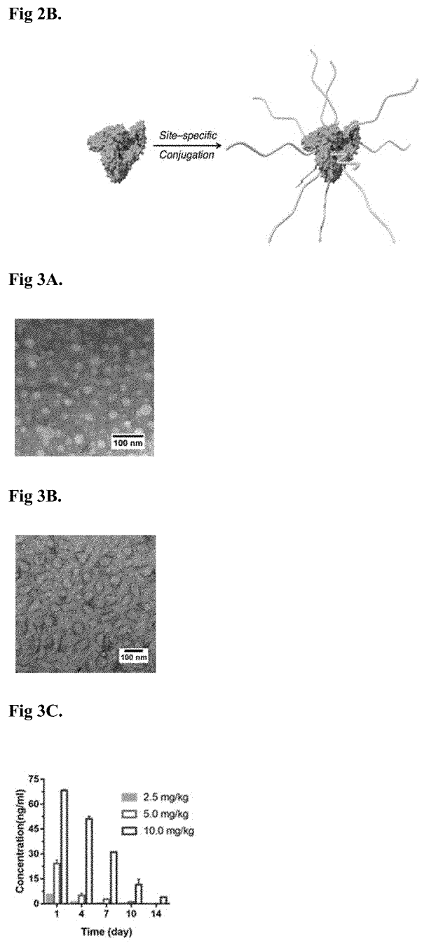

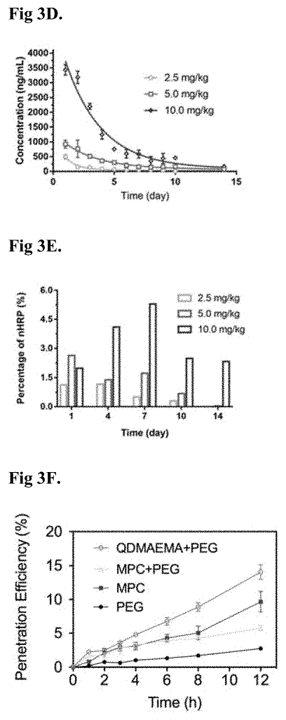

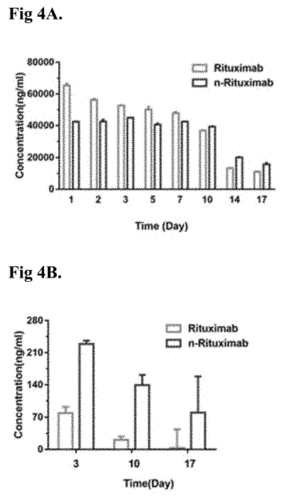

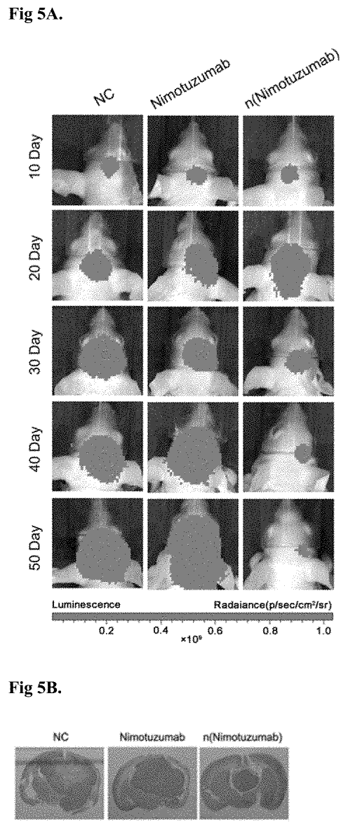

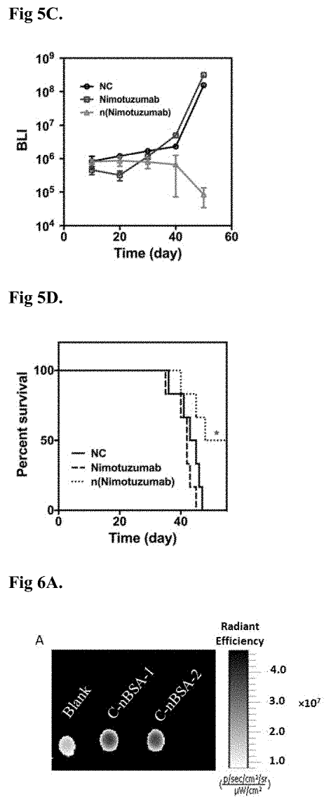

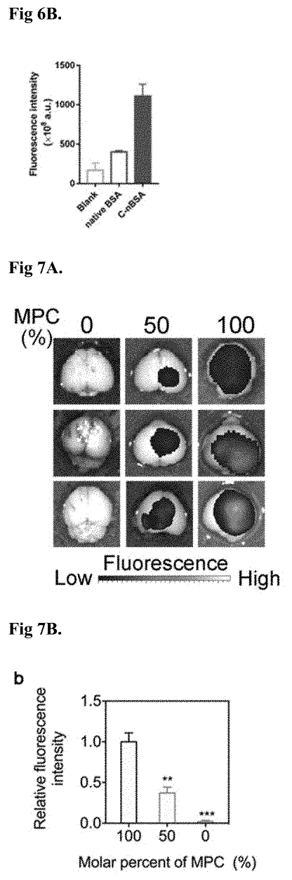

### DETAILED DESCRIPTION OF THE INVENTION

Certain molecules, such as nicotine, acetylcholine, meperidine, morphine, and cocaine may rapidly pass through the BBB to enter the brain. Choline, acetylcholine, and close analogues can cross the BBB through various transporters such as the choline transporters (ChT) and the nicotinic acetylcholine receptor (nAchR) (Lloyd, G. K. & Williams, J Pharmacol Exp Ther 292, 461-467 (2000)). 2-Methacryloyloxyethyl phosphorylcholine (MPC) and its polymer, poly(2-methacryloyloxyethyl phosphorylcholine) (PMPC) interact with the ChT or nAchR in a similar fashion to that of choline or acetylcholine. The transport of MPC and PMPC across the BBB is facilitated by ChT in a similar fashion to the transport of choline into the brain. Also, the transport of MPC and PMPC across the BBB is facilitated by nAchR through nAchR-mediated transcytosis.

Based on this design principle, a novel class of polymers that contain choline, acetylcholine, choline or acetylcholine analogues can be used to achieve effective CNS delivery via rapid transport across the BBB. Utilizing, these polymers, two major drug delivery systems can be envisaged, BBB-penetrative nanoparticles and BBB-penetrative polymer-bioactive agent conjugates.

Therapeutic proteins and genes can be encapsulated within a thin shell of polymer containing choline or acetyl choline analogues, which can be delivered to the CNS. A large variety of cross-linkers can be used to form the polymer shells, such as N,N′-methylenebisacrylamide (BIS), bis[2-(methacryloyloxy)ethyl] phosphate (BMEP), glycerol dimethacrylate (GDMA), polylactide-based block copolymers, and bisacrylated peptides. In certain embodiments, the corsslinker is degradable. The use of degradable cross-linkers allows the release of the proteins from the nanoparticles post degradation. The degradation of cross-linkers may result from hydrolysis reactions or degradation by tumor-specific proteases or matrix metalloproteases. Using nanoparticles with degradable linkers, therapeutic proteins can be released in the CNS upon penetration of the BBB.

BBB penetrative polymer-bioactive agent conjugates can be synthesized by conjugating therapeutics (e.g., proteins, genes) with polymers that contain the ligands for ChT or nAchRs. The molecular weight and chain structure of the polymers can be well controlled and functionalized with reactive moieties, allowing their effective conjugation with therapeutic proteins.

### Polymeric Nanoparticles

In one aspect, this invention provides polymeric nanoparticles comprising a cross-linked polymer shell, a plurality of bioactive agents encapsulated in the shell, and a plurality of transport moieties coupled to the polymer, wherein the transport moieties enhance penetration of the nanoparticle across the blood-brain barrier.

In certain embodiments, the polymer is a copolymer of transport monomers, each coupled to a transport moiety, and cross-linking monomers. In certain embodiments, the bioactive agent is not coupled to the copolymer. In certain embodiments, the copolymer further comprises stabilization monomers. In certain embodiments, the copolymer comprises agent monomers, each comprising an agent-association moiety that associates (non-covalently) with the agent.

In another aspect, this invention provides polymeric nanoparticles comprising a cross-linked polymer, a plurality of bioactive agents covalently coupled to the polymer, and a plurality of transport moieties coupled to the polymer, wherein the transport moieties enhance penetration of the nanoparticle across a blood brain barrier.

In certain embodiments, the copolymer further comprises a copolymer of transport monomers, each coupled to a transport moiety, and cross-linking monomers. In other embodiments, the copolymer further comprises a copolymer of transport monomers, hydrophilic monomers, each coupled to a hydrophilic moiety, and cross-linking monomers. In some embodiments, the polymer further comprises agent monomers, each coupled to a bioactive agent. In other embodiments, the copolymer comprises agent monomers, each comprising an agent-association moiety that associates (non-covalently) with the agent. In certain embodiments, the copolymer further comprises stabilization monomers.

Agent-association moieties are selected to associate with the agent under the conditions in which the polymerization is carried out. For example, for an agent that has a negatively-charged moiety or surface, positively charged agent-association moieties (e.g., amines, which are protonated to ammonium ions under neutral aqueous conditions) may be selected to foster association of the agent with the agent monomers under neutral aqueous conditions. Likewise, for an agent with a positively-charged moiety or surface, negatively-charged agent-association moieties (e.g., carboxylic acids, sulfonic acids, or phosphonic acids, which are deprotonated to anions under neutral aqueous conditions) may be selected to foster association of the agent with the agent monomers under neutral aqueous conditions. For non-polar agents, hydrophobic agent-association moieties (e.g., hydrophobic alkyl, aryl, or aralkyl groups) may be selected to foster association of the agent with the agent monomers under aqueous conditions.

Furthermore, the bioactive agents inside or on the surface of the nanoparticles are able to be fully released once the polymeric nanoparticle is degraded by an acid through hydrolysis, an esterase through enzymatic hydrolysis, or a protease through peptidase.

Importantly, by choosing and designing an appropriate polymer, the method of bioactive agent delivery to specific purposes (such as targeting by conjugating moieties to the polymer nanoparticles as described herein) can be modulated.

In certain embodiments, the polymer nanoparticles are 10 nm-20 nm, 20-25 nm, 25 nm-30 nm, 30 nm-35 nm, 35 nm-40 nm, 40 nm-45 nm, 45 nm-50 nm, 50 nm-55 nm, 55 nm-60 nm, 60 nm-65 nm, 70-75 nm, 75 nm-80 nm, 80 nm-85 nm, 85 nm-90 nm, 90 nm-95 nm, 95 nm-100 nm, or 100 nm-110 nm. In certain embodiments, the polymer nanoparticles are approximately 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 120 nm-130 nm, 130 nm-140 nm, 140 nm-150 nm, 150 nm-160 nm, 160 nm-170 nm, 170 nm-180 nm, 180 nm-190 nm, 190 nm-200 nm, 200 nm-210 nm, 220 nm-230 nm, 230 nm-240 nm, 240 nm-250 nm, or larger than 250 nm in diameter. In certain preferred embodiments, the polymer nanoparticles are about 5-20 nm in diameter.

In certain embodiments, the polymeric nanoparticle is about 10% cross-linked, about 20% cross-linked, about 30% cross-linked, about 40% cross-linked, about 50% cross-linked, about 60% cross-linked, about 70% cross-linked, about 80% cross-linked, about 90% cross-linked, or about 100% cross-linked.

In certain embodiments, the topology of the polymer is linear, dendritic, star-shaped, or hyperbranched.

In certain embodiments, polymer nanoparticles disclosed herein include non-targeting and targeting ability, higher efficiency, and/or lower adverse immune response. For example, the higher efficiency may result from increased uptake and more directed delivery.

In certain embodiments, the polymer nanoparticles are designed to degrade in 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 1 day, or 2 days, or 3 days, or 4 days or 5 days, or 6 days, or 1 week, or 2 weeks, or 3 weeks, or 1 month or any combination thereof. In certain embodiments, the polymer nanoparticles are designed to degrade at any of the above rates at a physiological pH. In certain embodiments, the polymer nanoparticles are designed to degrade at any of the rates above post-administration to a subject in need thereof. Generally speaking, the rate of degradation increases as the percentage of cross-linking increases, and the rate of degradation decreases as the percentage of cross-linking decreases. In other embodiments, the rate of degradation increases as the percentage of cross-linking increases. Accordingly, the percentage of cross-linking can be varied to affect the rate of degradation to achieve a desired degradation profile.

The enhanced stability of bioactive agents encapsulated by the polymeric nanoparticle or disposed upon the surface of the polymeric nanoparticle ensures its long-lasting circulation in body before it reaches the targeting sites. Overall, the delivery of bioactive agents as described herein can provide notable efficiency, augmented stability, and minimal toxicity.

### Bioactive Agents

In certain embodiments, the agent monomers are coupled to the bioactive agent by a linker. In certain embodiments, the linker is an alkyl chain, a heteroalkyl chain or an alkenyl chain. In certain embodiments, the linker comprises a plurality of linker monomers. In certain embodiments, the linker monomers are independently selected from N,N′-methylenebis(acrylamide) and bis[2-(methacryloyloxy)ethyl] phosphate (BMEP) monomers.

In certain embodiments, the linker is degradable. In certain embodiments, the degradable linker comprises a cleavable bond selected from an ester and a peptide. In certain embodiments, the degradable linker comprises a plurality of degradable linker monomers. In certain embodiments, the degradable linker monomers are selected from glycerol dimethacrylate, ethylene glycol dimethacrylate, and 2,2-bis(aminoethoxy)propane monomers.

In certain embodiments, linker is degradable by a protease, an esterase, an acid, a plasmin, a collagenase, and a matrix metalloprotease.

In certain embodiments, the bioactive agents are selected from small molecules, proteins, polynucleotides and imaging agents. In certain embodiments, the bioactive agents are selected from proteins, polynucleotides and imaging agents.

In certain embodiments, the bioactive agents are small molecules selected from antibiotic, antiviral, antineoplastic and antineurodegenerative agents.

In certain embodiments, the bioactive agents are small molecules selected from carmustine, lomustine, everolimus, temozolomide, paclitaxel, docetaxel, pemetrexed cisplatin, carboplatin, doxorubicin, cyclophosphamide, teniposide, mitomycin, irinotecan, vinorelbine, etoposide, ifosfamide, fluorouracil, prednisone, epirubicin, capecitabine, gemcitabine, ixabepilone, eribulin, pemetrexed, erlotinib, gefitinib, afatinib, crizotinib, ceritinib, alectinib, brigatinib, osimertinib, dabrafenib, tremetinib, sorafenib, sunitinib, temsirolimus, pazopanib, axitinib, cabozantinib, lenvatinib, leucovorin, oxaliplatin, irinotecan, regorafenib, trifluridine, tipiracil, cefaclor, cefradine, cefazolin, lincomycin, erythromycin, imipenem/cilastatin, oxacillin, cloxacillin, mecillinam, cefalexin, demeclocycline, doxycycline, minocycline, oxytetracycline, tetracycline, colistin, teicoplanin, gentamicin, kanamycin, neomycin, netilmicin, paromomycin, streptomycin, tobramycin, abacavir, didanosine, emtricitabine, lamivudine, stavudine, tenofovir disoproxil, zidovudine, efavirenz, etravirine, nevirapine, rilpivirine, atazanavir, darunavir, fosamprenavir, indinavir, nelfinavir, saquinavir, tipranavir, enfuvirtide, maraviroc, dolutegravir, elvitegravir, raltegravir, docosanol, acyclovir, valaciclovir, famciclovir, penciclovir, tacrine, rivastigmine, donepezil, galantamine, memantine, levodopa, bromocriptine, pergolide, pramipexole, ropinirole, piribedil, cabergoline, apomorphine, lisuride, safinamide, selegiline and rasagiline.

In certain embodiments, the bioactive agents are selected from growth factors, cytokines, antibodies, and enzymes. In certain embodiments, the bioactive agents are selected from bone morphogenetic proteins (BMP), epidermal growth factors (EGF), fibroblast growth factors (FGF), glial cell-derived neurotrophic factors (GDNF), interleukins (IL), nerve growth factors (NGF), brain-derived neurotrophic factors (BDNF), neurotrophins (NT), platelet-derived growth factors (PDGF), vascular endothelial growth factors (VEGF) and neuregulins. In certain embodiments, the bioactive agents are selected from bone morphogenetic protein 2 (BMP-2), bone morphogenetic protein 7 (BMP-7), epidermal growth factor, fibroblast growth factor 1 (FGF-1), fibroblast growth factor 2 (FGF-2), fibroblast growth factor 3 (FGF-3), fibroblast growth factor 4 (FGF-4), fibroblast growth factor 5 (FGF-5), fibroblast growth factor 6 (FGF-6), fibroblast growth factor 7 (FGF-7), fibroblast growth factor 8 (FGF-8), fibroblast growth factor 9 (FGF-9), fibroblast growth factor 10 (FGF-10), fibroblast growth factor 11 (FGF-11), fibroblast growth factor 12 (FGF-12), fibroblast growth factor 13 (FGF-13), fibroblast growth factor 14 (FGF-14), fibroblast growth factor 15 (FGF-15), fibroblast growth factor 16 (FGF-16), fibroblast growth factor 17 (FGF-17), fibroblast growth factor 18 (FGF-18), fibroblast growth factor 19 (FGF-19), fibroblast growth factor 20 (FGF-20), fibroblast growth factor 21 (FGF-21), fibroblast growth factor 22 (FGF-22), fibroblast growth factor 23 (FGF-23), glial cell-derived neurotrophic factor, neurturin, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), nerve growth factor, brain-derived neurotrophic factor, neurotrophin-3 (NT-3), neurotrophin 4 (NT-4), platelet-derived growth factor, vascular endothelial growth factor, neuregulin 1 (NRG1), neuregulin (NRG2), neuregulin (NRG3), and neuregulin (NRG4).

In certain embodiments, the bioactive agents are selected from Afutuzumab, 3F8, 8H9, Adecatumumab, Abituzumab, ado-trastuzumab emtansine, Altumomab pentetate, Atezolizumab Glembatumumab vedotin, Margetuximab, Sacituzumab govitecan, Trastuzumab emtansine, Lorvotuzumab mertansine, Anetumab ravtansine, Ascrinvacumab, Avelumab, Azintuxizumab vedotin, bevacizumab, bevacizumab-awwb, BCD-100, Belantamab mafodotin, Bectumomab, Belimumab, Bemarituzumab, Brontictuzumab, Brentuximab vedotin, Cantuzumab ravtansine, Carotuximab, Cabiralizumab, cBR96-doxorubicin immunoconjugate, Camidanlumab tesirine, Cemiplimab, cetuximab, Cetrelimab, Cibisatamab, Citatuzumab bogatox, Cixutumumab, Codrituzumab, Cofetuzumab pelidotin, Coltuximab ravtansine, Conatumumab, Cusatuzumab, Depatuxizumab mafodotin, Dacetuzumab, Dalotuzumab, Detumomab, Daratumumab, Demcizumab, Derlotuximab biotin, Denintuzumab mafodotin, Emapalumab, Bivatuzumab mertansine, Carlumab, Clivatuzumab tetraxetan, Dinutuximab, Dostarlimab, Drozitumab, DS-8201, Durvalumab, Dusigitumab, Duvortuxizumab, Ecromeximab, Eculizumab, Edrecolomab, Elgemtumab, Elotuzumab, Emactuzumab, Emibetuzumab, Enapotamab vedotin, Enavatuzumab, Enfortumab vedotin, Enoblituzumab, Ensituximab, Epratuzumab, Ertumaxomab, Etaracizumab, Farletuzumab, FBTA05, Ficlatuzumab, Figitumumab, Flanvotumab, Flotetuzumab, Fresolimumab, Futuximab, Galiximab, Gancotamab, Ganitumab, Gatipotuzumab, Gemtuzumab ozogamicin, Girentuximab, Glembatumumab vedotin, IBI308, Ibritumomab tiuxetan, Icrucumab, Igovomab, Iladatuzumab vedotin, IMAB362, Imalumab, Imgatuzumab, Indatuximab ravtansine, Indusatumab vedotin, Inebilizumab, Intetumumab, Inotuzumab ozogamicin, Ipilimumab, Iratumumab, Isatuximab, Istiratumab, Labetuzumab, Lacnotuzumab, Ladiratuzumab vedotin, Lenzilumab, Lexatumumab, Lifastuzumab vedotin, Loncastuximab tesirine, Losatuxizumab vedotin, Lilotomab satetraxetan, Lintuzumab, Lirilumab, Lorvotuzumab mertansine, Lucatumumab, Lumiliximab, Lumretuzumab, MABp1, Mapatumumab, Margetuximab, Matuzumab, Milatuzumab, Mirvetuximab soravtansine, Mitumomab, Modotuximab, Mogamulizumab, Monalizumab, Mosunetuzumab, Moxetumomab pasudotox, Nacolomab tafenatox, Naptumomab estafenatox, Narnatumab, Navicixizumab, Naxitamab, Necitumumab, Nesvacumab, Nimotuzumab, Nivolumab, Nofetumomab merpentan, Obinutuzumab, Ocaratuzumab, Ofatumumab, Olaratumab, Oleclumab, Omburtamab, Onartuzumab, Ontuxizumab, Oportuzumab monatox, Oregovomab, Otlertuzumab, Pascolizumab, Pasotuxizumab, Pateclizumab, Patritumab, PDR001, Pembrolizumab, Pemtumomab, Pertuzumab, Pidilizumab, Pinatuzumab vedotin, Pintumomab, Polatuzumab vedotin, Pritumumab, Racotumomab, Radretumab, Ramucirumab, Relatlimab, Rilotumumab, Rituximab, Robatumumab, Rosmantuzumab, Rovalpituzumab tesirine, Sacituzumab govitecan, Samalizumab, Samrotamab vedotin, Satumomab pendetide, Seribantumab, Sibrotuzumab, SGN-CD19A, Siltuximab, Sirtratumab vedotin, Sofituzumab vedotin, Solitomab, Spartalizumab, Tabalumab, Tacatuzumab tetraxetan, Taplitumomab paptox, Tarextumab, Tavolimab, Telisotuzumab vedotin, Tenatumomab, Tepoditamab, Tetulomab, TGN1412, Tigatuzumab, Timigutuzumab, Tiragotumab, Tislelizumab, Tisotumab vedotin, TNX-650, Tomuzotuximab, Tositumomab, Tovetumab, Trastuzumab, Trastuzumab emtansine, TRBS07, Tremelimumab, Tucotuzumab celmoleukin, Ublituximab, Ulocuplumab, Urelumab, Utomilumab, Vadastuximab talirine, Vandortuzumab vedotin, Vantictumab, Vanucizumab, Varlilumab, Veltuzumab, Vesencumab, Volociximab, Vonlerolizumab, Vorsetuzumab mafodotin, Votumumab, XMAB-5574, Zalutumumab, Zanolimumab, Zatuximab, Zenocutuzumab, Zolbetuximab, Aducanumab, Bapineuzumab, Crenezumab, Elezanumab, Eptinezumab, Erenumab, Erlizumab, Fremanezumab, Fulranumab, Foravirumab, Galcanezumab, Gantenerumab, Gosuranemab, Ibalizumab, Lampalizumab, Larcaviximab, Ozanezumab, Pamrevlumab, Panitumumab, Pankomab, Placulumab, Ponezumab, Prasinezumab, Porgaviximab, PRO 140, Ranibizumab, Refanezumab, Rinucumab, Rafivirumab, Rmab, SA237, Satralizumab, Solanezumab, Sonepcizumab, Stamulumab, Suvizumab, Tanezumab, Tefibazumab, Teprotumumab, Trevogrumab, Varisacumab antibodies.

In certain embodiments, the bioactive agents are selected from α-L-iduronidase, iduronate-2-sulfatase, heparin N-sulfatase, glucocerebrosidase, galactocerebrosidase, arylsulfatase A, β-hexosaminidase, recombinant tripeptidyl peptidase 1, α-galactosidase A, tripeptidyl peptidase 1 proenzyme, and α-glucosidase enzymes.

In certain embodiments, the bioactive agents are selected from gendicine, advexin, oblimersen sodium, miR-15a, miR-16-1, miR-143, miR-145, miR-21, members of the let-7 family, miR-142, BIC/miR-155, a member of the miR-17-19b cluster, glutamate decarboxylate (GAD), netrin (NTN), artermin, aromatic L-amino acid decarboxylase (AADC), ciliary neurotrophic factor (CNTF), insulin like growth factor 1 (IGF-1), vascular endothelial growth factor A (VEGF-A), vascular endothelial growth factor B (VEGF-B), survival of motor neuron 1 (SMN1), survival of motor neuron 2 (SMN2), and chemokine receptor type 5 (CCR5).

In certain embodiments, the bioactive agents are selected from DNA with an endosome-escaping protein (e.g., phospholipase), RNA with an endosome-escaping protein (e.g., phospholipase), a CRISPR/Cas 9 complex, a RNA-induced silencing complex (RISC), and a microRNA protein-RNA complex (RNP).

In certain embodiments, the bioactive agents are selected from radio-nucleotides, radiolabeled glucose residues, radiolabeled antibodies, radiolabeled proteins, and fluorescent proteins.

The radio-imaging agents of the invention may be used in accordance with the methods of the invention by those of skill in the art, e.g., by specialists in nuclear medicine, to image tissue in a mammal. Any mammalian tumor may be imaged the imaging agents of the invention. Images are generated by virtue of differences in the spatial distribution of the imaging agents which accumulate in the various tissues and organs of the mammal. The spatial distribution of the imaging agent accumulated in a mammal, in an organ, or in a tissue may be measured using any suitable means, for example, a PET or single photon emission computer tomography (SPECT) imaging camera apparatus, and the like.

### Targeted Delivery

Generally, the targeting moieties described herein serve to target or direct the nanoparticle or polymer-bioactive agent conjugate to a specific site (e.g., cell type, or diseased tissue) or interaction (e.g., a specific binding event). Of particular interest, the polymeric nanoparticles and polymer-bioactive agent conjugates can be used to target the brain and the central nervous system. In certain embodiments, a nanoparticle or polymer-bioactive agent conjugate comprising the targeting moiety is delivered to a specific site (e.g., ischemic tissue) more effectively than a nanoparticle or polymer-bioactive agent conjugate lacking the targeting moiety.

In certain embodiments, bioactive agents can be effectively delivered to specific sites in vivo through the use of the targeting moieties. In certain embodiments, targeted delivery of bioactive agents into cells is achieved using surface-conjugated targeting moieties on optimized nanoparticles.

In certain embodiments, the conjugation prevents the dissociation of the targeting agent from the polymer nanoparticle.

In certain embodiments, the invention is practiced using non-targeted and targeted polymer nanoparticles for bioactive agent delivery with high efficiency and low toxicity for in vitro testing and in vivo targeting to specific tissues and organs via intravenous injection.

In certain embodiments, the polymeric nanoparticle further comprises a plurality of targeting moieties. In certain embodiments, the targeting moieties are disposed on the surface of the polymeric nanoparticle. In certain embodiments, the targeting moieties are covalently coupled to the nanoparticle.

In certain embodiments, the polymers of the polymer-bioactive agent conjugate further comprise a plurality of targeting moieties. In certain embodiments, the targeting moieties are covalently coupled to the polymers.

In certain embodiments, the targeting moieties are selected from a targeting small molecules, and targeting proteins.

In certain embodiments, the targeting small molecules are selected from folate and arginylglycylaspartic acid (RGD).

In certain embodiments, the targeting proteins are transferrins.

In certain embodiments, the targeting proteins are selected from an anti-VEGFR antibody, an anti-CD20 antibody and an anti-ERBB2 antibody.

In certain embodiments, the targeting protein is selected from an antigen binding fragment (Fab), a single-chain variable fragment (ScFv) and a disulfide-stabilized variable antibody fragment (ds-Fv).

### Polymer-Bioactive Agents Conjugates

In another aspect, this invention provides polymer-bioactive agent conjugates that comprise a bioactive agent covalently coupled to a plurality of polymers, and a plurality of transport moieties coupled to the polymers. In certain embodiments, each polymer comprises a copolymer of transport monomers, each coupled to a transport moiety, and agent monomers, each coupled to the bioactive agent.

In certain embodiments, the polymers comprise a plurality of monomers independently selected from 2-methacryloyloxyethyl phosphorylcholine (MPC), N-(3-aminopropyl) methacrylamide (APm), trimethyl(2-prop-2-enoyloxyethyl)azanium, methacrylatoethyl trimethyl ammonium, [2-(methacryloyloxy)ethyl]dimethyl-(3-sulfopropyl)ammonium hydroxide, carboxybetaine methacrylate, acrylamide, poly(ethylene glycol) methyl ether acrylate, vinyl pyridine, and carboxybetaine acrylamide monomers. In certain embodiments, the polymers comprise a plurality of monomers independently selected from 2-methacryloyloxyethyl phosphorylcholine (MPC), N-(3-aminopropyl) methacrylamide (APm), trimethyl(2-prop-2-enoyloxyethyl)azanium, methacrylatoethyl trimethyl ammonium, [2-(methacryloyloxy)ethyl]dimethyl-(3-sulfopropyl)ammonium hydroxide, carboxybetaine methacrylate and carboxybetaine acrylamide monomers. In certain embodiments, the polymers comprise a plurality of monomers independently selected from 2-methacryloyloxyethyl phosphorylcholine and a plurality of N-(3-aminopropyl) methacrylamide monomers.

In certain embodiments, the bioactive agent is selected from a growth factor, a cytokine, an antibody, an enzyme, a gene, a gene-protein complex and an imaging agent. In certain embodiments, the bioactive agent is selected from a bone morphogenetic protein (BMP), a epidermal growth factor (EGF), a fibroblast growth factor (FGF), a glial cell-derived neurotrophic factor (GDNF), an interleukin (IL), a nerve growth factor (NGF), a brain-derived neurotrophic factor (BDNF), a neurotrophin (NT), a platelet-derived growth factor (PDGF), a vascular endothelial growth factor (VEGF) and a neuregulin. In certain embodiments, the bioactive agent is selected from bone morphogenetic protein 2 (BMP-2), bone morphogenetic protein 7 (BMP-7), epidermal growth factor, fibroblast growth factor 1 (FGF-1), fibroblast growth factor 2 (FGF-2), fibroblast growth factor 3 (FGF-3), fibroblast growth factor 4 (FGF-4), fibroblast growth factor 5 (FGF-5), fibroblast growth factor 6 (FGF-6), fibroblast growth factor 7 (FGF-7), fibroblast growth factor 8 (FGF-8), fibroblast growth factor 9 (FGF-9), fibroblast growth factor 10 (FGF-10), fibroblast growth factor 11 (FGF-11), fibroblast growth factor 12 (FGF-12), fibroblast growth factor 13 (FGF-13), fibroblast growth factor 14 (FGF-14), fibroblast growth factor 15 (FGF-15), fibroblast growth factor 16 (FGF-16), fibroblast growth factor 17 (FGF-17), fibroblast growth factor 18 (FGF-18), fibroblast growth factor 19 (FGF-19), fibroblast growth factor 20 (FGF-20), fibroblast growth factor 21 (FGF-21), fibroblast growth factor 22 (FGF-22), fibroblast growth factor 23 (FGF-23), glial cell-derived neurotrophic factor, neurturin, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), nerve growth factor, brain-derived neurotrophic factor, neurotrophin-3 (NT-3), neurotrophin 4 (NT-4), platelet-derived growth factor, vascular endothelial growth factor, neuregulin 1 (NRG1), neuregulin (NRG2), neuregulin (NRG3), and neuregulin (NRG4).

In certain embodiments, the bioactive agent is selected from Afutuzumab, 3F8, 8H9, Adecatumumab, Abituzumab, ado-trastuzumab emtansine, Altumomab pentetate, Atezolizumab Glembatumumab vedotin, Margetuximab, Sacituzumab govitecan, Trastuzumab emtansine, Lorvotuzumab mertansine, Anetumab ravtansine, Ascrinvacumab, Avelumab, Azintuxizumab vedotin, bevacizumab, bevacizumab-awwb, BCD-100, Belantamab mafodotin, Bectumomab, Belimumab, Bemarituzumab, Brontictuzumab, Brentuximab vedotin, Cantuzumab ravtansine, Carotuximab, Cabiralizumab, cBR96-doxorubicin immunoconjugate, Camidanlumab tesirine, Cemiplimab, cetuximab, Cetrelimab, Cibisatamab, Citatuzumab bogatox, Cixutumumab, Codrituzumab, Cofetuzumab pelidotin, Coltuximab ravtansine, Conatumumab, Cusatuzumab, Depatuxizumab mafodotin, Dacetuzumab, Dalotuzumab, Detumomab, Daratumumab, Demcizumab, Derlotuximab biotin, Denintuzumab mafodotin, Emapalumab, Bivatuzumab mertansine, Carlumab, Clivatuzumab tetraxetan, Dinutuximab, Dostarlimab, Drozitumab, DS-8201, Durvalumab, Dusigitumab, Duvortuxizumab, Ecromeximab, Eculizumab, Edrecolomab, Elgemtumab, Elotuzumab, Emactuzumab, Emibetuzumab, Enapotamab vedotin, Enavatuzumab, Enfortumab vedotin, Enoblituzumab, Ensituximab, Epratuzumab, Ertumaxomab, Etaracizumab, Farletuzumab, FBTA05, Ficlatuzumab, Figitumumab, Flanvotumab, Flotetuzumab, Fresolimumab, Futuximab, Galiximab, Gancotamab, Ganitumab, Gatipotuzumab, Gemtuzumab ozogamicin, Girentuximab, Glembatumumab vedotin, IBI308, Ibritumomab tiuxetan, Icrucumab, Igovomab, Iladatuzumab vedotin, IMAB362, Imalumab, Imgatuzumab, Indatuximab ravtansine, Indusatumab vedotin, Inebilizumab, Intetumumab, Inotuzumab ozogamicin, Ipilimumab, Iratumumab, Isatuximab, Istiratumab, Labetuzumab, Lacnotuzumab, Ladiratuzumab vedotin, Lenzilumab, Lexatumumab, Lifastuzumab vedotin, Loncastuximab tesirine, Losatuxizumab vedotin, Lilotomab satetraxetan, Lintuzumab, Lirilumab, Lorvotuzumab mertansine, Lucatumumab, Lumiliximab, Lumretuzumab, MABp1, Mapatumumab, Margetuximab, Matuzumab, Milatuzumab, Mirvetuximab soravtansine, Mitumomab, Modotuximab, Mogamulizumab, Monalizumab, Mosunetuzumab, Moxetumomab pasudotox, Nacolomab tafenatox, Naptumomab estafenatox, Narnatumab, Navicixizumab, Naxitamab, Necitumumab, Nesvacumab, Nimotuzumab, Nivolumab, Nofetumomab merpentan, Obinutuzumab, Ocaratuzumab, Ofatumumab, Olaratumab, Oleclumab, Omburtamab, Onartuzumab, Ontuxizumab, Oportuzumab monatox, Oregovomab, Otlertuzumab, Pascolizumab, Pasotuxizumab, Pateclizumab, Patritumab, PDR001, Pembrolizumab, Pemtumomab, Pertuzumab, Pidilizumab, Pinatuzumab vedotin, Pintumomab, Polatuzumab vedotin, Pritumumab, Racotumomab, Radretumab, Ramucirumab, Relatlimab, Rilotumumab, Rituximab, Robatumumab, Rosmantuzumab, Rovalpituzumab tesirine, Sacituzumab govitecan, Samalizumab, Samrotamab vedotin, Satumomab pendetide, Seribantumab, Sibrotuzumab, SGN-CD19A, Siltuximab, Sirtratumab vedotin, Sofituzumab vedotin, Solitomab, Spartalizumab, Tabalumab, Tacatuzumab tetraxetan, Taplitumomab paptox, Tarextumab, Tavolimab, Telisotuzumab vedotin, Tenatumomab, Tepoditamab, Tetulomab, TGN1412, Tigatuzumab, Timigutuzumab, Tiragotumab, Tislelizumab, Tisotumab vedotin, TNX-650, Tomuzotuximab, Tositumomab, Tovetumab, Trastuzumab, Trastuzumab emtansine, TRBS07, Tremelimumab, Tucotuzumab celmoleukin, Ublituximab, Ulocuplumab, Urelumab, Utomilumab, Vadastuximab talirine, Vandortuzumab vedotin, Vantictumab, Vanucizumab, Varlilumab, Veltuzumab, Vesencumab, Volociximab, Vonlerolizumab, Vorsetuzumab mafodotin, Votumumab, XMAB-5574, Zalutumumab, Zanolimumab, Zatuximab, Zenocutuzumab, Zolbetuximab, Aducanumab, Bapineuzumab, Crenezumab, Elezanumab, Eptinezumab, Erenumab, Erlizumab, Fremanezumab, Fulranumab, Foravirumab, Galcanezumab, Gantenerumab, Gosuranemab, Ibalizumab, Lampalizumab, Larcaviximab, Ozanezumab, Pamrevlumab, Panitumumab, Pankomab, Placulumab, Ponezumab, Prasinezumab, Porgaviximab, PRO 140, Ranibizumab, Refanezumab, Rinucumab, Rafivirumab, Rmab, SA237, Satralizumab, Solanezumab, Sonepcizumab, Stamulumab, Suvizumab, Tanezumab, Tefibazumab, Teprotumumab, Trevogrumab, Varisacumab.

In certain embodiments, the bioactive agent is selected from α-L-iduronidase, iduronate-2-sulfatase, heparin N-sulfatase, glucocerebrosidase, galactocerebrosidase, arylsulfatase A, β-hexosaminidase, recombinant tripeptidyl peptidase 1, α-galactosidase A, tripeptidyl peptidase 1 proenzyme, and α-glucosidase.

In certain embodiments, the bioactive agent is selected from gendicine, advexin, oblimersen sodium, miR-15a, miR-16-1, miR-143, miR-145, miR-21, a member of the let-7 family, miR-142, BIC/miR-155, a member of the miR-17-19b cluster, GAD, NTN, artermin AADC, CNTF, IGF-1, VEGF-A, VEGF-B, SMN1 cDNA, an antisense oligonucleotide restoring inclusion of SMN2 exon 7, and CCR5.

In certain embodiments, the bioactive agent is selected from a DNA with an endosome-escaping protein (e.g., phospholipase), a RNA with an endosome-escaping protein (e.g., phospholipase), a CRISPR/Cas 9 complex, a RNA-induced silencing complex (RISC), and a microRNA protein-RNA complex (RNP).

In certain embodiments, the bioactive agent is a fluorescent protein.

### Enhanced Penetration of Biological Membranes

The polymeric nanoparticles and polymer-bioactive agent conjugates of the invention comprise a plurality of transport moieties that enhance penetration of the nanoparticle or conjugate across biological membranes. In certain embodiments, the transport moieties enhance the penetration of the nanoparticle or conjugate across a blood-brain barrier when compared to a nanoparticle or conjugate that lacks said transport moieties.

In general, the transport moieties (e.g., Formula I, V, or VI) are each coupled to a transport monomer, which in turn are coupled to the copolymer. Alternatively, the transport moiety itself is a monomer and it is directly coupled to the copolymer (e.g., compounds of Formula II, III, IV, or VI). In certain embodiments, the transport moieties are independently selected from a neurotransmitter, an opioid and a central nervous system stimulant. In certain embodiments, the transport moieties may be covalently coupled to the polymer. In other embodiments, the transport moieties may be non-covalently (e.g., via hydrogen bonding or charge-charge interactions) coupled to the polymer.

In certain embodiments, the transport moieties are independently selected from choline, acetylcholine, nicotine, phosphorylcholine, muscarine, cocaine, meperidine, morphine, dopamine and serotonin moieties.

In certain embodiments, the transport moiety is represented by Formula I, II, III, IV, V, VI, or a pharmaceutically acceptable salt thereof:

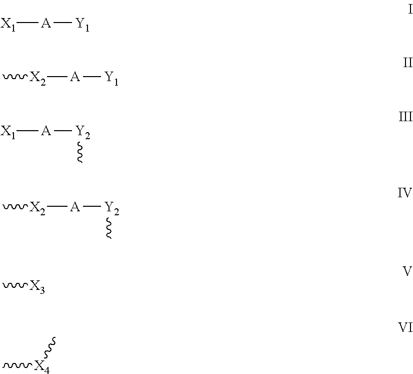

wherein:
X₁, X₂, X₃, or X₄ is a cationic moiety, preferably comprising nitrogen;
A is a spacer unit;
Y₁ or Y₂ is a hydrogen bond acceptor, preferably containing nitrogen or oxygen; and
[symbol] represents a bond to the polymer.

In certain embodiments, the cationic moiety has at least one positive charge or at least two positive charges.

In certain embodiments, the hydrogen bond acceptor has at least one hydrogen bond acceptor, at least two hydrogen bond acceptors, or at least three hydrogen bond acceptors.

In certain embodiments, the transport moiety is represented by Formula I or a pharmaceutically acceptable salt thereof,

X₁-A-Y₁ I

wherein
X₁ is amino, guanidino, hydrazino, diazonium, phosphonium, sulfonium, or heterocyclyl;
A is alkyl, aryl, cycloalkyl, or heteroaryl; and
Y₁ is amido, amidino, oxygen, alkyloxy, azo, carboxyl, alkenyl, ester, keto, phosphate, sulfoxide, sulfone, sulfonamido, heteroaryl, or heterocyclyl.

In certain embodiments, the transport moiety is represented by Formula II or a pharmaceutically acceptable salt thereof,

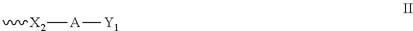

wherein
X₂ is amino, guanino, hydrazino, phosphonium, sulfonium, or heterocyclyl;
A is alkyl, aryl, cycloalkyl, or heteroaryl; and
Y₁ is amido, amidino, oxygen, alkyloxy, azo, carboxyl, alkenyl, ester, keto, phosphate, sulfoxide, sulfone, sulfonamido, heteroaryl, or heterocyclyl.

In certain embodiments, Y₁ is alkenyl. In certain embodiments, Y₁ is

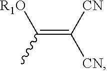

wherein R₁ is hydrogen, alkyl, aryl, heteroaryl, or heterocyclyl.

In certain embodiments, the transport moiety is represented by Formula III or a pharmaceutically acceptable salt thereof,

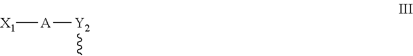

wherein
X₁ is amino, guanidino, hydrazino, diazonium, phosphonium, sulfonium, or heterocyclyl;
A is alkyl, aryl, cycloalkyl, or heteroaryl; and
Y₂ is amido, amidino, oxygen, alkoxy, azo, carboxyl, alkenyl, ester, keto, phosphate, sulfoxide, sulfone, sulfonamido, heteroaryl, or heterocyclyl.

In certain preferred embodiments of Formula I, II, or III, A is alkyl. In certain, even more preferred embodiments, A is C₁-C₁₀alkyl.

In certain embodiments, the transport moiety is represented by Formula IV or a pharmaceutically acceptable salt thereof,

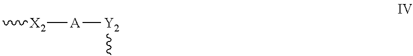

wherein
X₂ is amino, guanidino, hydrazino, phosphonium, sulfonium, or heterocyclyl;
A is alkyl, aryl, cycloalkyl, or heteroaryl; and
Y₂ is amido, amidino, oxygen, alkoxy, azo, carboxyl, alkenyl, ester, keto, phosphate, sulfoxide, sulfone, sulfonamido, heteroaryl, or heterocyclyl.

In certain embodiments, the transport moiety is represented by Formula V or a pharmaceutically acceptable salt thereof,

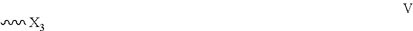

wherein
X₃ is amino, guanidino, hydrazino, diazonium, phosphonium, sulfonium, or heterocyclyl.

In certain embodiments, the transport moiety is represented by Formula VI or a pharmaceutically acceptable salt thereof,

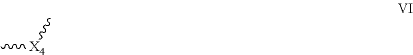

wherein
X₄ is amino, guanidino, hydrazino, phosphonium, sulfonium, or heterocyclyl.

In certain embodiments of Formula I, II, III, IV, V, or VI, the amino moiety is a tertiary or quaternary amino moiety.

In certain embodiments, the transport moieties enhance the penetration of the nanoparticle or conjugate across biological membranes when compared to a nanoparticle or conjugate that lacks the transport moieties.

In certain embodiments, the polymeric nanoparticles and polymer-bioactive agent conjugates of the invention comprise at least one hydrophilic moiety that improves the penetration of the nanoparticle or conjugate across biological membranes and improves blood plasma stability when compared to a nanoparticle or conjugate that lacks the hydrophilic moieties.

In general, the hydrophilic moieties improve the blood plasma stability and penetration of the nanoparticle or conjugate across biological membranes by reducing the adhesion of organic matter and biomolecules present in the blood. In some embodiments, the hydrophilic moieties may be small hydrophilic molecules (e.g., monomers), oligomers, or polymers that are attached to the surface of the conjugate or nanoparticle once the particle or conjugate has been synthesized. In other embodiments, the hydrophilic moieties may be incorporated into the nanoparticle at the time of synthesis (e.g., the moieties are coupled to hydrophilic monomers make up the copolymer). In certain embodiments, the hydrophilic moieties are covalently coupled to the polymer. In certain embodiments, the hydrophilic moieties are enmeshed or embedded in the polymer. In certain embodiments, the hydrophilic moieties are non-covalently (e.g., via hydrogen bonding) coupled to the polymer.

In certain embodiments, the hydrophilic moiety comprises at least one hydrogen bond acceptor (e.g., nitrogen or oxygen).

In certain embodiments, the hydrophilic moiety is a zwitterionic polymer. In certain embodiments, the zwitterionic polymer is a poly(phosphorylcholine), a poly(sulfobetaine), or a poly(carboxybetaine).

In other embodiments, the hydrophilic moiety is not a zwitterionic polymer. In certain embodiments, the hydrophilic moiety is a neutral polymer. In certain embodiments, the hydrophilic moiety is poly(ethylene glycol), poly(vinylpyridine), poly(2-hydroxyethyl methacrylate), or a poly-saccharide. In certain embodiments, the hydrophilic moiety is a poly(oligoethylene glycol) poly(methacrylate), poly(acrylate), poly(amide), poly(peptoid), poly(oxazoline), poly(hydroxylethylacrylate), and poly(ethyl ethylene phosphate).

In certain embodiments the molar ratio of hydrophilic moieties to transport moieties is about 1:1, about 2:1, about 3:1, about 4:1, about 5:1, about 10:1, or about 20:1. In certain embodiments, the ratio of hydrophilic moieties to transport moieties is about 1:1. In certain embodiments, the ratio of hydrophilic moieties to transport moieties is about 20:1.

In certain embodiments, the hydrophilic moieties are present in an amount such that, upon exposure to a quantity of biomolecules, surfaces of the polymeric nanoparticles or polymer-bioactive agent conjugate adsorb less than about 50%, less than about 40%, less than about 30%, less than about 20%, less than about 10%, less than about 5%, less than about 4%, less than about 3%, less than about 2%, or less than about 1% by number of the biomolecules, as compared to the number of biomolecules adsorbed by the surface of the polymeric nanoparticles or polymer-bioactive agent conjugate alone, without the hydrophilic moiety, under identical or substantially the same conditions.

### Monomers and Cross-Linkers

Different monomers and cross-linkers can be used to deliver the bioactive agents (e.g., proteins, small molecules, genes) by in situ polymerization, such as those taught in this specification and others well known in the art.

In certain embodiments, the transport monomers are independently selected from 2-methacryloyloxyethyl phosphorylcholine (MPC), N-(3-aminopropyl) methacrylamide (APm), trimethyl(2-prop-2-enoyloxyethyl)azanium, quaternized dimethylaminoethyl methacrylate, methacrylatoethyl trimethyl ammonium, [2-(Methacryloyloxy)ethyl]dimethyl-(3-sulfopropyl)ammonium hydroxide, a carboxybetaine methacrylate, and carboxybetaine acrylamide monomers. In a preferred embodiment the transport monomers are methacryloyloxyethyl phosphorylcholine monomers. In certain embodiments, the transport monomers comprise vinyl pyridine monomers. In certain embodiments, the transport monomers comprise poly(ethylene glycol) methyl ether acrylate monomers.

In certain embodiments, the agent monomers are methacrylate esters that are covalently coupled to one or more bioactive agents, e.g., an ester formed with a hydroxyl on the bioactive agent itself, or with a hydroxyl on a linker covalently coupled to one or more bioactive agents. Representative examples include methacrylate esters of polyethylene glycol, methacrylate esters of small molecules (e.g., a methacrylate ester of everolimus) and methacrylate esters of antibodies and proteins containing peptides with a hydroxyl moiety e.g., serine and threonine.

In certain embodiments, the agent monomers are methyacrylamides that are covalently coupled to one or more bioactive agents, e.g., an amide formed with a amine on the bioactive agent itself, or with an amine on a linker covalently coupled to one or more bioactive agents. Representative examples include methyacrylamides of small molecules (e.g., a methyacrylamide of fluoxetine) and methyacrylamides of antibodies and proteins containing peptides with an amino moiety e.g., lysine. In certain embodiments, the agent monomers are independently selected from methacrylate ester, methyacrylamide and N-(3-aminopropyl) methacrylamide (APm) monomers

In certain embodiments, the cross-linking monomers are independently selected from N,N′-methylenebisacrylamide (BIS), bis[2-(methacryloyloxy)ethyl] phosphate (BMEP), glycerol dimethacrylate (GDMA), a polylactide-based block copolymer and a bisacrylated peptide. In certain embodiments, the cross-linking monomers are independently selected from glycerol dimethacrylate, a polylactide-based block copolymer, and a bisacrylated peptide. In certain embodiments, the polylactide-based block copolymer is poly(DL-lactide)-b-Poly(ethylene glycol)-b-Poly(DL-lactide)-diacrylate. In certain embodiments, the bisacrylated peptide is bisacrylated VPLGVRTK. In certain embodiments, the bisacrylated peptide is bisacrylated KNRVK. In certain embodiments, the bisacrylated peptide is bisacrylated GGIPVSLRSGGK. In certain embodiments, the bisacrylated peptide is bisacrylated GGVPLSLYSGGK. In certain embodiments, the bisacrylated peptide is a substrate for an extracellular protease.

In certain embodiments, the stabilization monomers are polyethylene glycol monomers.

In certain embodiments, the cross-linking monomers are degradable by an acid, a base, an enzyme, adenosine triphosphate, an oxidant, a reductant, glucose, hypoxic conditions, visible light, ultra violet light, infrared light, or heat. In certain embodiments, the cross-linking monomers are degradable by a protease, an esterase, a plasmin, a collagenase, and a matrix metalloprotease.

Some exemplary degradable cross-linkers and non-degradable cross-linkers are listed in Table 2 of US 2015/0071999.

### Methods of Imaging

In certain embodiments, the invention provides a method of imaging the central nervous system of a subject in need thereof, comprising administering to the subject an effective amount of a polymeric nanoparticle or polymer-bioactive agent of any one of the preceding claims, wherein the polymeric nanoparticle or polymer-bioactive agent comprises an imaging agent. In certain embodiments, the method further comprises imaging the subject by detecting the imaging agent.

### Methods of Treatment

In certain embodiments, the invention is used treat a disease or disorder selected from a cancer, a neurodegenerative disease, a central nervous system disorder and a viral infection.

In certain embodiments, the cancer is selected from blastoma, glioblastoma, myeloma, neoplasm of the central nervous system (CNS), tumor angiogenesis, spinal axis tumor, brain stem glioma, pituitary adenoma, and environmentally induced cancers.

In certain embodiments, the neurodegenerative disease or central nervous system disorder is selected from Alzheimer's disease, Parkinson's disease, Dementia with Lewy bodies, Multiple system atrophy, Prion diseases, Motor neuron disease, Huntington's disease, Amyotrophic lateral sclerosis, Ataxia telangiectasia, Pick's disease, Krabbe's disease, Kennedy's disease, Primary lateral sclerosis, Cockayne syndrome, Metachromatic leukodystrophy, Tay Sach's disease, Sandhoff's disease, Late infantile neuronal ceroid lipofuscinosis, Pompe's disease, Spinocerebellar ataxia, HIV-associated neurocognitive disorders, Stroke, Lou Gehrig's disease, Creutzfeldt-Jakob disease, Spinal cord injury, Cerebral palsy, Multiple sclerosis, Progressive supranuclear palsy, Pelizaeus-Merzbacher disease, Tabes dorsalis, and Spinal muscular atrophy.

In certain embodiments, the neurodegenerative disease or central nervous system disorder is cerebral ischemia.

In certain embodiments, the viral infection is selected from human immunodeficiency virus (HIV), Rabies virus, Zika virus, and Herpes simplex virus (HSV).

Non-limiting examples of cellular proliferative and/or differentiative disorders include cancer, e.g., carcinoma, blastoma, glioblastoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplastic cells of hematopoietic origin, e.g., arising from myeloid, lymphoid or erythroid lineages, or precursor cells thereof.

### Methods of Administration

The pharmaceutical compositions encompassed by the invention may be administered by any suitable means including, but not limited to oral or parenteral routes, including intravenous, intramuscular, intraperitoneal, and subcutaneous administration. In certain embodiments, the pharmaceutical compositions are administered by intravenous or intraparenteral infusion or injection. In preferred embodiments, the pharmaceutical compositions are administered by an intravenous injection. In other preferred embodiments, the polymeric nanoparticle or polymer-bioactive agent is administered topically (e.g., in the form of eye drops).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, pheasants, and/or turkeys. Preferred subjects are humans.

In certain embodiments, the disclosed compositions are nasally administered in the range of once per day, to once per week, to once per two weeks, to once per month. In certain embodiments, the nanoparticles and compositions are administered nasally. As used herein, the term “nasally” or “nasal administration” refers to a delivery of the nanoparticles to the mucosa of the subject's nose such that the nanoparticles content is absorbed directly into the nasal tissue.

In certain embodiments, the polymer nanoparticles and polymer-bioactive agents conjugates, and compositions comprising them are for systemic administration.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the nanoparticle or conjugate is preferably administered as a pharmaceutical composition comprising, for example, a nanoparticle or conjugate of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutically acceptable carrier can contain physiologically acceptable agents that act, for example, to stabilize, increase solubility or to increase the absorption of a nanoparticle or polymer-bioactive agent of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a nanoparticle or conjugate of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); and subcutaneously. In certain embodiments, a nanoparticle or conjugate may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the nanoparticle or conjugate which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a nanoparticle or conjugate, such as a nanoparticle or conjugate of the invention, with the carrier and, optionally, one or more accessory ingredients. In general, the formulations are prepared by uniformly and intimately bringing into association a nanoparticle or conjugate of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a nanoparticle or conjugate of the present invention as an active ingredient. Compositions, nanoparticle or conjugate may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinylpyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered nanoparticle or conjugate, moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the nanoparticle or conjugate, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active nanoparticle or conjugate may be mixed under sterile conditions with a pharmaceutically acceptable carrier, and with any preservatives, buffers, or propellants that may be required.

The ointments, pastes, creams and gels may contain, in addition to an active nanoparticle or conjugate, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more nanoparticle or conjugate in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or degradable devices. Various slow release polymeric devices have been developed and tested in vivo in recent years for the controlled delivery of drugs, including proteinaceous biopharmaceuticals. A variety of biocompatible polymers (including hydrogels), including both degradable and non-degradable polymers, can be used to form an implant for the sustained release of a nanoparticle or conjugate at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular nanoparticle or conjugate or combination of nanoparticles or conjugates employed, or the ester, salt or amide thereof, the route of administration, the time of administration, the rate of excretion of the particular nanoparticle(s) or conjugate(s) being employed, the duration of the treatment, other drugs, compounds and/or materials used in combination with the particular nanoparticle(s) or conjugate(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition, nanoparticle or conjugate at levels lower than that required in order to achieve the desired therapeutic effect and gradually increase the dosage until the desired effect is achieved. By “therapeutically effective amount” is meant the concentration of a nanoparticle or conjugate that is sufficient to elicit the desired therapeutic effect. It is generally understood that the effective amount of the nanoparticle or conjugate will vary according to the weight, sex, age, and medical history of the subject. Other factors which influence the effective amount may include, but are not limited to, the severity of the patient's condition, the disorder being treated, the stability of the nanoparticle or conjugate, and, if desired, another type of therapeutic agent being administered with the nanoparticle or conjugate of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active nanoparticle or conjugate used in the compositions and methods of the invention will be that amount of the nanoparticle or conjugate that is the lowest dose effective to produce a therapeutic effect. Such an effective dose will generally depend upon the factors described above.

If desired, the effective daily dose of the nanoparticle or conjugate may be administered as one, two, three, four, five, six or more sub-doses administered separately at appropriate intervals throughout the day, optionally, in unit dosage forms. In certain embodiments, of the present invention, the nanoparticle or conjugate may be administered two or three times daily. In preferred embodiments, the nanoparticle or conjugate will be administered once daily.

The patient receiving this treatment is any animal in need, including primates, in particular humans; and other mammals such as equines, cattle, swine, sheep, cats, and dogs; poultry; and pets in general.

In certain embodiments, nanoparticles or conjugates of the invention may be used alone or conjointly administered with another type of therapeutic agent.

The present disclosure includes the use of pharmaceutically acceptable salts of nanoparticles or conjugates of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Synthesis

In one aspect, the present disclosure provides a method of making a polymeric nanoparticle comprising: combining i) a plurality of transport monomers, each coupled to at least one transport moiety; ii) a plurality of cross-linking monomers; iii) a plurality of agent monomers, each coupled to at least one bioactive agent; and iv) a solvent; and initiating copolymerization of the monomers with an initiator. In certain embodiments, combining further comprises combining with a plurality of targeting moieties. In certain embodiments, combining further comprises combining with a plurality of stabilization monomers.

In another aspect, the present disclosure provides a method of making a polymeric nanoparticle comprising combining i) a plurality of transport monomers, each coupled to at least one transport moiety; ii) a plurality of cross-linking monomers; iii) a plurality of bioactive agents; and iv) a solvent; and initiating copolymerization of the monomers with an initiator. In certain embodiments, the bioactive agents are selected from proteins, polynucleotides and imaging agents. In certain embodiments, the bioactive agents and monomers are in a molar ratio of about 5,000:1 to 12,000. In certain embodiments, the bioactive agents and monomers are in a molar ratio of about 5,000:1. In certain embodiments, the bioactive agents and monomers are in a molar ratio of about 12,000:1. In certain embodiments, the bioactive agents and monomers are in a molar ratio of about 500:1. In certain embodiments, combining further comprises combining with a plurality of targeting moieties. In certain embodiments, combining further comprises combining with a plurality of stabilization monomers. In certain embodiments, combining further comprises combining with a plurality of hydrophilic moieties.

In another aspect, the present disclosure provides a method of making a polymeric nanoparticle comprising: combining i) a plurality of cross-linking monomers; ii) a plurality of agent monomers, each coupled to at least one bioactive agent; and iii) a solvent; and initiating copolymerization of the monomers with an initiator. In certain embodiments, combining further comprises combining with a plurality transport moieties. In certain embodiments, combining with a plurality of transport moieties comprises combining with a plurality of transport monomers, each coupled to at least one transport moiety. In certain embodiments, combining further comprises combining with a plurality of targeting moieties. In certain embodiments, combining further comprises combining with a plurality of stabilization monomers.

In another aspect, the present disclosure provides a method of making a polymeric nanoparticle comprising: combining i) a plurality of transport monomers, each coupled to at least one transport moiety; ii) a plurality of cross-linking monomers; iii) a solvent; and initiating copolymerization of the monomers with an initiator. In certain embodiments, combining further comprises combining with a plurality of bioactive agents. In certain embodiments, combining with a plurality of bioactive agents comprises combining with a plurality of agent monomers, each coupled to at least one bioactive agent. In certain embodiments, combining further comprises combining with a plurality of targeting moieties. In certain embodiments, combining further comprises combining with a plurality of stabilization monomers.

In certain embodiments, the initiator is selected from a free radical initiator, a nucleophilic initiator and an electrochemical initiator.

In certain embodiments, the methods of synthesis disclosed herein further comprise at least one purification step. In certain embodiments, the purification step is centrifugation. In certain embodiments, the purification step is filtration.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

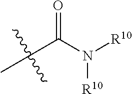

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

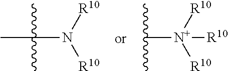

wherein each R¹⁰ independently represents a hydrogen or a hydrocarbyl group, or two R¹⁰ are taken together with the N atom to which they are attached complete a heterocycle having from 4 to 8 atoms in the ring structure.

The term “amidino”, is an art recognized term, and refers to the group represented by the general formulae

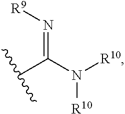

wherein R⁹ and R¹⁰ each represent substituents such as hydrogen or a hydrocarbyl group, such as alkyl, aryl, heteroaryl, cycloalkyl, or heterocyclyl.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

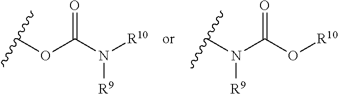

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The term “guanidino”, is an art recognized term, and refers to the group represented by the general formulae

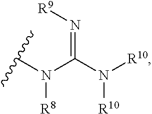

wherein R⁸, R⁹, and R¹⁰ each represent substituents such as hydrogen or a hydrocarbyl group, such as alkyl, aryl, heteroaryl, cycloalkyl, or heterocyclyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “hydrazino”, is an art recognized term, and refers to the group represented by the general formulae

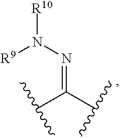

wherein R⁹ and R¹⁰ each represent substituents such as hydrogen or a hydrocarbyl group, such as alkyl, aryl, heteroaryl, cycloalkyl, or heterocyclyl.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “phosphate”, is an art recognized term, and refers to the group represented by the general formulae

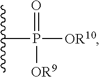

wherein R⁹ and R¹⁰ each represent substituents such as hydrogen or a hydrocarbyl group, such as alkyl, aryl, heteroaryl, cycloalkyl, or heterocyclyl.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “silyloxy” refers to an oxygen moiety with a silyl attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

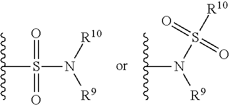

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

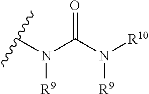

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethyl silyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, the terms “degradable” and “nondegradable” refer to the ability of the polymers described herein to degrade into smaller fragments. In certain embodiments, of this invention, degradable polymers can break down at physiological pH. In certain embodiments, degradable polymers can break down at approximately pH 7.4. In certain embodiments, a mixture of degradable and nondegradable polymers can yield a degradable polymer mixture. In certain embodiments, a mixture of degradable and nondegradable polymers can break down at physiological pH. In certain embodiments, a mixture of degradable and nondegradable polymers can break down at approximately pH 7.4.

Examples of positively charged monomers, cross-linkers, and neutral monomers are provided in Table 1, Table 2, and Table 3 of U.S. Patent Application publication no. US 2015/0071999, which is herein incorporated by reference in its entirety.

As used herein, the term “targeting moiety” refers to a moiety (e.g., an antibody, a hormone, a hormone derivative, a folic acid, a gene, a folic acid derivative, a biotin, a small molecule, an oligopeptide, a sigma-2-ligand, or a sugar) that serves to target or direct the conjugate to a particular location (e.g., cell type, or diseased tissue) or interaction (e.g., a specific binding event).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

As used herein, the term “Bioactive agents” refers to a substance which may be used in connection with an application that is therapeutic or diagnostic, such as, for example, in methods for diagnosing the presence or absence of a disease in a patient and/or methods for the treatment of a disease in a patient. “Bioactive agents” refers to substances which are capable of exerting a biological effect in vitro and/or in vivo. The bioactive agents may be neutral, positively or negatively charged. Suitable bioactive agents include, for example, prodrugs, imaging agent, diagnostic agents, therapeutic agents, pharmaceutical agents, drugs, oxygen delivery agents, blood substitutes, synthetic organic molecules, proteins, peptides, vitamins, steroids, steroid analogs and genetic material, including nucleosides, nucleotides and polynucleotides.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds for laboratory use, or for subsequent conversion to a pharmaceutically acceptable acid addition salt.

The term “pharmaceutically acceptable basic addition salt” as used herein means any non-toxic organic or inorganic base addition salt of any acid compounds. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments, of the present invention, and are not intended to limit the invention.

### Example 1: Compounds Delivery of BBB-Penetrative Protein Nanoparticles to CNS

Horseradish peroxide was first conjugated with N-acryloxysuccinimide (NAS) to attach acryloyl groups onto their surfaces. The average number of acryloyl groups conjugated onto a protein molecule was determined by measuring the residual (unreacted) lysine on the protein molecule with a fluoresamine assay was found to be approximately 4.5.

Following acryloxylation, the proteins were encapsulated using in situ polymerization method. MPC and cross linker bis-methacrylamide (BIS) were first prepared as 40% (m/v) in DI water and 10% (m/v) stock solution in anhydrous DMSO, respectively. Then MPC and BIS were added into the solution of HRP proteins (1 mg/mL) being encapsulated at a molar ratio of 5000:1 (MPC to HRP proteins) and 500:1 (BIS to HRP proteins), respectively. Polymerization was initiated by the addition of APS (300:1) and TEMED (1200:1) and kept at 4° C. for 2 h. After the polymerization, the solution was dialyzed against phosphate buffer solution (PBS) to remove unreacted monomers and by-products. The horseradish peroxide nanoparticles (nHRP) were further purified with a hydrophobic interaction column (Phenyl-Sepharose CL-4B) to remove any un-encapsulated horseradish peroxide. nHRP were examined using transmission electron microscopy (TEM), which showed a uniform spherical shape with an average diameter of ˜30 nm.

HRP nanoparticles were administrated to non-human primates (rhesus macaque monkey) at doses of 2.5, 5.0 and 10.0 mg/kg via intravenous injection. Post the intravenous injection, the plasma was collected every day for 2 weeks, and the cerebrospinal fluid (CSF) was collected at day 1, 4, 7, 10 and 14. nHRP exhibited a prolonged circulation time in the plasma, which could be detected 2 weeks after the injection. The cerebral spinal fluid (CSF) collected after the first day post the intravenous injection of 10 mg/kg n-HRP was examined using TEM, which showed the presence of particles with morphology and size similar those of nHRP. As the CSF was almost protein-free and the particle size was much larger than that of blood proteins, the nanoparticles shown in the TEM image of the CSF sample were the nHRP crossing the BBB to the CSF. This study clearly indicates a successful delivery of n-HPR to the CSF.

The concentrations of n-HRP in plasma and CSF were quantified by a TMB substrate Kit (Thermo Fisher Scientific, USA). In the 1^{st} day, the concentration of nHRP in the CSF of rhesus macaque monkeys injected with 2.5 mg/kg, 5.0 mg/kg, and 10.0 mg/kg n-HRP were 5.57 ng/mL, 24.17 ng/mL, and 68.21 ng/mL, which are equivalent to 1.12%, 2.63%, and 1.99% of the concentration of nHRP in the plasma, respectively. This indicates that nHRP was delivered into CSF in a dose-dependent manner. The concentration of nHRP in the CSF reached ˜5.3% of that in the plasma concentration at day 4 post the injection.

### Example 2: Delivery of Therapeutic Proteins to the CNS of a Monkey

Rituximab proteins were directly encapsulated via in situ polymerization without a acryloxylation process. MPC was used as the monomer; poly(DL-lactide)-b-Poly(ethylene glycol)-b-Poly(DL-lactide)-diacrylate triblock copolymers (PLA-PEG-PLA diac) and glycerol dimethacrylate (GDMA) were used as the degradable cross linker. MPC, PLA-PEG-PLA copolymer and GDMA were added into the solution of Rituximab proteins (2.2 mg/mL) at a molar ratio of 12000:1 (MPC to protein), 500:1 (PLA-PEG-PLA copolymer to protein) and 500:1 (GDMA to protein). The polymerization was initiated by the addition of APS (2000:1) and TEMED (8000:1) at 4° C. for 3 h. After the polymerization, the solution was concentrated with the centrifugal filter units to remove unreacted monomers and by-products. Protein nanoparticles were further purified with hydrophobic interaction column (Phenyl-Sepharose CL-4B) to remove un-encapsulated proteins.

To evaluate the CNS delivery efficiency, Rituximab nanoparticles were administrated to non-human primates (rhesus macaque monkey) at a dose of 5.0 mg/kg via intravenous injection. After intravenous administration, plasma was collected at 1, 2, 3, 5, 7, 10, 14 and 17 days post injection, and CSF was collected at day 3, 10 and 17. Both Rituximab and Rituximab nanoparticles (n-Rituximab) exhibit similarly long circulation time in the plasma of rhesus macaque monkeys. The monkey administrated with native Rituximab showed significantly lower CSF concentration than that with n-Rituximab (5-15 fold higher). High concentration of Rituximab was observed in CSF for the monkey administered with n-Rituximab 17 days after a single intravenous injection. In sharp contrast, Rituximab in CSF was undetectable by week 2 for the monkey administrated with native Rituximab.

### Example 3: Treatment of Brain Tumors in a Mouse

Nanoparticles of Nimotuzumab, (n(Nimotuzumab)), were synthesized using MPC and peptide cross-linkers with an amino acid sequence of VPLGVRTK, which can be degraded by tumor protease.

Nimotuzumab solution (5 mg/mL) was diluted to 1 mg/mL using phosphate buffers (20 mM, pH=7.4) under ice-bath. N-(3-aminopropyl) methacrylamide (APm), prepared in a 100 mg/mL aqueous solution, was added into the protein solution with stirring for 10 min at 4° C. APm was enriched around Nimotuzumab through electrostatic and hydrophobic interactions. 2-Methacryloyloxyethyl phosphorylcholine and bisacryloylated VPLGVRTK peptide were added to protein solution sequentially with rapid stirring. The molar ratio of MPC:APm:crosslinker was adjusted to 50:5:1. Radical polymerization was initiated by adding both ammonium persulfate (1:10 molar ratio of total monomers) dissolved in deionized water and the same volume of 10% (w/v) N,N,N′,N′-tetramethylethylenediamine into the reaction solution. The polymerization was allowed to proceed for 90-120 mins in a nitrogen atmosphere at 4° C. Finally, unreacted monomers, cross-linkers, and initiators were removed by dialysis in PBS (20 mM, pH=6.5). After dialysis, n(Nimotuzumab) were passed through a Sephadex G-200 columns by gravity to remove un-encapsulated Nimotuzumab antibodies.

To create an intracranial tumor model, U87-EGFRwt cells were infected with luciferase lentivirus (Genepharma, Shanghai, China). After a 2-day infection, 5×10⁵ cells were collected and injected into the intracranial striatum of 5-week-old female nu/nu-nude mice with a stereotactic instrument. The mice were treated with native Nimotuzumab or n(Nimotuzumab) (5 mg/kg body weight, i.v. injection) every other day until 9 doses had been given. Treatments were initiated 3 days after tumor cell injection. In control group, each animal was injected i.v. with 100 μl of sterile PBS. Each group has 9 mice. To acquire tumor growth status in live animals of different treatment groups by bioluminescent imaging, the mice were anesthetized, injected intraperitoneally with 50 mg/mL of D-luciferin (Promega, China), and imaged with the IVIS Imaging System (Caliper Life Sciences) for 10-120 s.

Tumors in mice treated by n(Nimotuzumab) show significantly suppressed progression compared to those in the control group and the native-Nimotuzumab-treated group. After the last injection (day 20), tumor-bearing brain tissues from each group were collected to ultimately confirm the therapeutic efficacy of n(Nimotuzumab). HE staining results show that n(Nimotuzumab) treatment exhibited significant tumor growth reduction compared to native Nimotuzumab treatment.

### Example 4: Delivery of Protein Conjugates

Protein conjugates were synthesized by conjugating polymer, poly(2-methacryloyloxyethyl phosphorylcholine) (PMPC), to fluorescence-labeled bovine serum albumin (BSA). The BSA conjugates were injected to Balb/c mice via intravenously at a dose of 5.0 mg/kg. One date after the intravenous injection, fluorescence intensity of the mouse brains was compared. The mice administrated with the conjugates displayed 6 times higher fluorescence intensity than mice injected with PBS, and 4 times higher fluorescence intensity than mice injected with native BSA.

### Example 5: Treatment of Brain Tumors in a Mouse

The effectiveness of delivering nimotuzumab to brain tumors is attributed to the MPC that contains choline and acetylcholine analogues. For validation, a series of nimotuzumab nanocapsules were synthesized by replacing 0%, 50% and 100% of the MPC monomer with vinyl pyridine, a monomer used to synthesize nanocapsules with long circulative half-life but without the BBB-penetration ability. The series of nanocapsules contain different contents of MPC while exhibiting similar size and surface charge. FIGS. 7A and 7B depict the in-vivo bioluminescent images of the orthotopic U87-EGFRwt glioma xenograft mice and the ex-vivo fluorescent images of the collected brain tissues 4 hours after intravenous administration of such nanocapsules. Mice brains with similarly sized tumors were treated with nanocapsules made with 100% MPC, displayed a 2.7 fold increase in the fluorescent intensity compared to those with nanocapsules made with 50% MPC. No brain uptake is observed for the mice with the nanocapsules made with 0% MPC. This observation indicates that the MPC enables the effective delivery of n(Nimo) to the brain tumor.

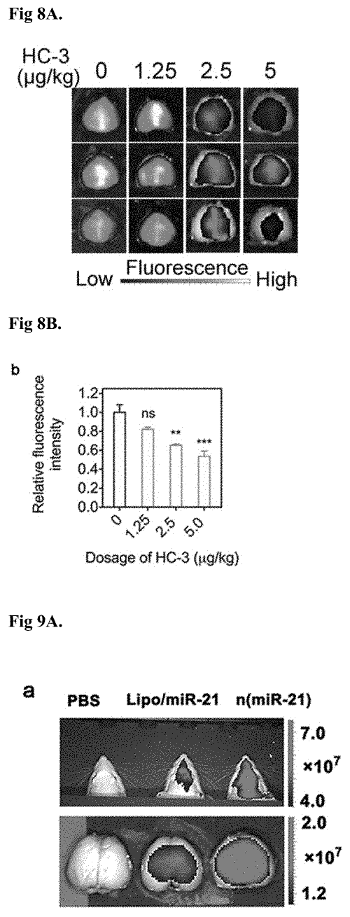

The BBB penetration of the nanocapsules can be attributed to their transcytosis mediated by the choline and acetylcholine receptors. Glioma-bearing mice were intraperitoneally injected with 1.25, 2.5 or 5 μg/kg of choline-transporter inhibitor, hemicholinium-3 (HC-3), 20 mins prior to the injection of Cy5.5-labeled n(Nimo). FIGS. 8A and 8B depict the in-vivo bioluminescent images of the orthotopic U87-EGFRwt glioma xenograft mice and the ex-vivo fluorescent images of the brains 4 hours after injection. Increasing the dose of HC-3 significantly reduces the fluorescent intensity of the glioma-bearing brain, with no significant difference in both tumor size and body weight, indicating that the choline transporter mediates the transport of n(Nimo) into the brain. This method provides an effective antibody delivery strategy for brain tumor treatment.

### Example 6: Treatment of Cerebral Ischemia in a Rat

Acrylamide (AAm), N-(3-Aminopropyl) methacrylamide (APm) and poly(ethylene glycol) methyl ether acrylate with an average molecular weight of 2000 Da (mPEG) were prepared as a 10% (w/v) stock solution in deoxygenated RNase-free water. A solution of glycerol dimethacrylate (GDMA) was prepared as a 10% (w/v) stock solution in anhydrous DMSO. Then specific amounts of above monomers and crosslinkers were added to the miRNA solution, and the molar ratio of AAm/APm/mPEG/GDMA was tuned for screening of synthetic parameters. Polymerization was initiated by the addition of ammonium persulfate (1/10 molar ratio of total monomers) and N,N,N′,N′-tetramethylethylenediamine (2-fold weight ratio of APS) and kept at 4° C. for 2 h. The final miRNA concentration was tuned to 5 μM by diluting with deoxygenated RNase-free water. After polymerization, the solution was dialyzed against 10 mM PBS using a 10 kDa membrane to remove unreacted monomers and by-products. Non-PEG n(miR-21) was synthesized by a similar protocol without the use of mPEG.

The transient focal cerebral ischemia rat model was induced by middle cerebral artery occlusion reperfusion (MCAO/R). See Liu et al. (2013) Biomaterials 34, 817-830. SD rats were anesthetized with 10% chloralic hydras (350 mg/kg, intraperitoneally (i.p.)). Body temperature was monitored and maintained at 37° C. A midline incision was made on the ventral side of the neck and muscles were gently pulled aside; then, the right common carotid artery and the junction of internal and external carotid artery were dissected carefully. The external carotid artery was ligated and cauterized. A surgical nylon monofilament (diameter 0.234 mm) with its tip rounded by heating near a flame was inserted into the internal carotid artery through a nick of the external carotid stump to block the origin of middle cerebral artery. After 1 hour of ischemia, the filament was pulled out for reperfusion.

The in vivo application of the miRNA nanocapsules for brain ischemia therapy was demonstrated in a rat model of transient focal cerebral ischemia induced by middle cerebral artery occlusion reperfusion (MCAO/R). MiR-21 labeled with Cy5.5 (Cy5.5-miR-21) was used to synthesize Lipo/miR-21 and n(miR-21). Then, these two samples were injected intravenously 1 day after ischemia injury at a dosage of 0.5 mg/kg miR-21, respectively. Accumulation of the samples in both ischemic brains were analyzed using both fluorescence imaging and qRT-PCR 24 hours after administration.

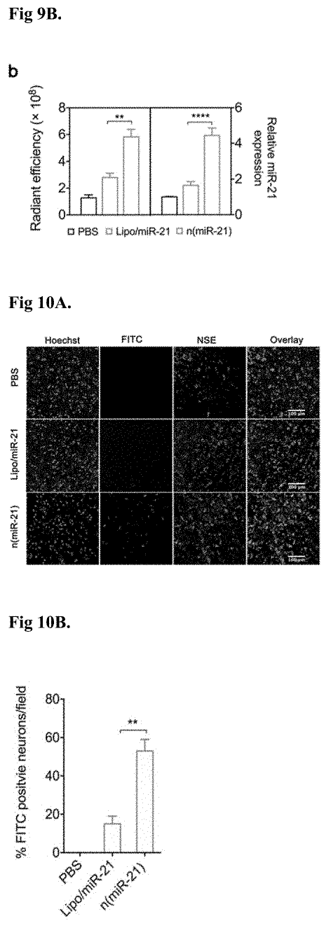

Ischemic rats administrated with n(miR-21) showed a stronger fluorescent intensity in the head and isolated brain tissues than those with Lipo/miR-21 in (FIG. 9A). The radiant efficiency of miR-21 delivered by n(miR-21) or Lipo/miR-21 was quantitatively compared in both ischemic and non-ischemic rats. As shown in FIG. 9B, the radiant efficiency of n(miR-21) was 2-fold and 3.5-fold higher than that of Lipo/miR-21 and PBS control in the ischemic rats, respectively, indicating more efficient delivery of n(miR-21) to the ischemic brain tissue. The miR-21 expressions in ischemic brain tissues after delivery were further compared by qRT-PCR (FIG. 9B). The level of miR-21 in the brains of ischemic rats treated by n(miR-21) was 2.7-fold greater in comparison than those treated by Lipo/miR-21, indicating n(miR-21) enhances the regulation in the ischemic brain tissues in comparison with Lipo/miR-21.

FITC-labeled miR-21 was used to further understand the distribution of n(miR-21) in the ischemic brain tissues. The ischemic hemisphere of brain tissues was isolated, fixed, and stained 24 hours after injection. The fluorescence signal from n(miR-21) was much stronger than that from Lipo/miR-21, further approving the improved delivery efficiency of miRNA by nanocapsules in vivo (FIG. 10A). Moreover, the distribution of fluorescence signals from n(miR-21) matched the patterns of the cell nuclei staining and neuron marker staining. The delivery efficiency in neuron cells was quantitatively and specifically analyzed in FIG. 10B. A significant improvement of neuron delivery was shown with n(miR-21) compared to Lipo/miR-21. The miR-21 expressions in brain tissues after delivery were further compared by qRT-PCR. The level of miR-21 in the brains from rats treated by n(miR-21) was 2.7-fold greater in comparison with those treated by Lipo/miR-21.

Neurological scores were assessed 24 hours after MCAO/R for the confirmation of successful building of transient focal cerebral ischemia model. See Liu et al. (2013) Biomaterials 34, 817-830. An examiner blinded to the experimental groups performed behavior assessments. Neurological deficit was scored based on the following description: 0, no deficits; 1, difficulty in fully extending the contralateral forelimb; 2, unable to extend the contralateral forelimb; 3, mild circling to the contralateral side; 4, severe circling and 5, falling to the contralateral side. Rats with neurologic deficit scores ranged from 1-3 were selected as the model. After neurological scores assess, rats were lethally anesthetized, and brain slices was harvested for HE staining to confirm the successful building of transient focal cerebral ischemia model.

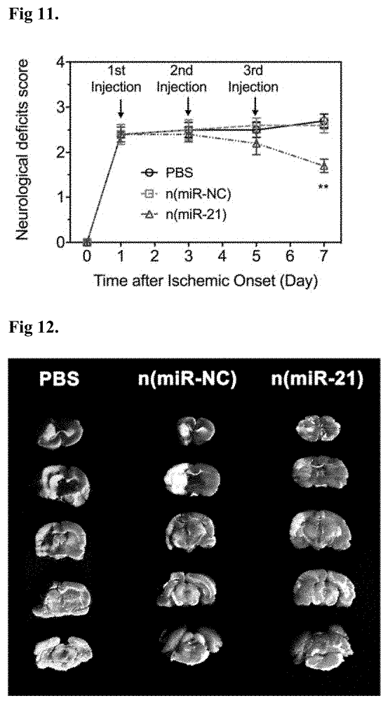

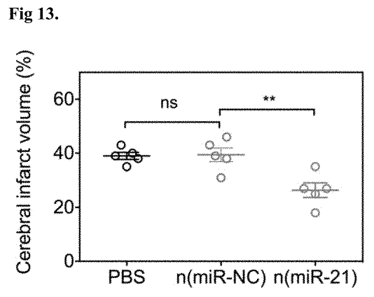

As depicted in FIG. 11, the average neurological deficit scores of the rats treated with either PBS or n(miR-NC) only slightly improves with time; while those treated with n(miR-21) show remarkably improved performance. Brain tissues were collected at day 7 to assess the infarct volume by 2,3,5-triphenyltetrazolium hydrochloride (TTC) staining. As depicted in FIGS. 12 and 13, the TTC-stained brains of the n(miR-21)-treated rats exhibited significantly decreased infarct volume (˜45%) compared to those treated with PBS or n(miR-NC).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments, of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A polymeric nanoparticle comprising a cross-linked polymer matrix, a plurality of bioactive agents encapsulated in the matrix, and a plurality of transport moieties coupled to the polymer, wherein the transport moieties enhance penetration of the nanoparticle across a blood brain barrier.

2. The polymeric nanoparticle of claim 1, wherein the bioactive agent is not covalently coupled to the copolymer.

3. The polymeric nanoparticle of claim 2, wherein the copolymer further comprises agent monomers, each agent monomer having an agent-association moiety that non-covalently associates with the agent, and the agent:
i) comprises a positively-charged moiety and the agent monomer comprises a negatively-charged agent-association moiety;
ii) comprises a negatively-charged moiety and the agent monomer comprises a positively-charged agent-association moiety; or
iii) is hydrophobic and the agent monomer comprises a hydrophobic agent-association moiety.

4-6. (canceled)

7. The polymeric nanoparticle of claim 1, wherein the bioactive agent is covalently coupled to the copolymer.

8. The polymeric nanoparticle of claim 1, wherein if the bioactive agent is a protein then the polymer matrix does not comprise 2-methacryloyloxyethyl phosphorylcholine (MPC) and N,N′-methylenebisacrylamide (BIS).

9. (canceled)

10. The polymeric nanoparticle of claim 7, wherein either:
i) the transport moieties are covalently coupled to the polymer; or
ii) the transport moieties are non-covalently coupled to the polymer.

11. (canceled)

12. The polymeric nanoparticle of claim 1, wherein the transport moieties comprise moieties selected from neurotransmitters, opioids central nervous system stimulants, choline, acetylcholine, nicotine, phosphorylcholine, muscarine, cocaine, meperidine, morphine, dopamine and serotonin moieties.

13. (canceled)

14. The polymeric nanoparticle of claim 1, wherein the transport moiety is represented by Formula I, II, III, IV, V, VI, or a pharmaceutically acceptable salt thereof: 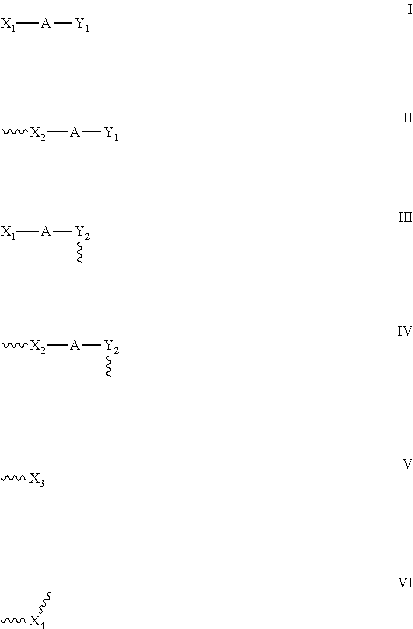
wherein:
X₁, X₂, X₃, or X₄ is a cationic moiety; preferably comprising nitrogen;
A is a spacer unit;
Y₁ or Y₂ is a hydrogen bond acceptor preferably containing nitrogen or oxygen; and [symbol]
represents a bond to the polymer.

15. (canceled)

16. (canceled)

17. The polymeric nanoparticle of claim 14, wherein the transport moiety is represented by Formula I or a pharmaceutically acceptable salt thereof,
X₁-A-Y₁ I
wherein
X₁ is amino, guanidino, hydrazino, diazonium, phosphonium, sulfonium, or heterocyclyl;
A is alkyl, aryl, cycloalkyl, or heteroaryl; and
Y₁ is amido, amidino, oxygen, alkoxy, azo, carboxyl, alkenyl, ester, keto, phosphate, sulfoxide, sulfone, sulfonamido, heteroaryl, or heterocyclyl.

18. The polymeric nanoparticle of claim 17, wherein Y₁ is alkenyl or 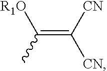
wherein R₁ is hydrogen, alkyl, aryl, heteroaryl, or heterocyclyl.

19. (canceled)

20. The polymeric nanoparticle of claim 14, wherein the transport moiety is represented by Formula II or a pharmaceutically acceptable salt thereof, 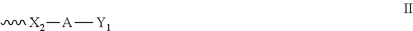
wherein
X₂ is amino, guanidino, hydrazino, phosphonium, sulfonium, or heterocyclyl;
A is alkyl, aryl, cycloalkyl, or heteroaryl; and
Y₁ is amido, amidino, oxygen, alkyloxy, azo, carboxyl, alkenyl, ester, keto, phosphate, sulfoxide, sulfone, sulfonamido, heteroaryl, or heterocyclyl.

21. The polymeric nanoparticle of claim 14, wherein the transport moiety is represented by Formula III or a pharmaceutically acceptable salt thereof, 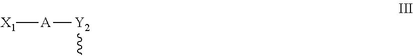
wherein
X₁ is amino, guanidino, hydrazino, diazonium, phosphonium, sulfonium, or heterocyclyl;
A is alkyl, aryl, cycloalkyl, or heteroaryl; and
Y₂ is amido, amidino, oxygen, alkoxy, azo, carboxyl, alkenyl, ester, keto, phosphate, sulfoxide, sulfone, sulfonamido, heteroaryl, or heterocyclyl.

22. The polymeric nanoparticle of claim 14, wherein the transport moiety is represented by Formula IV or a pharmaceutically acceptable salt thereof, 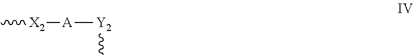
wherein
X₂ is amino, guanidino, hydrazino, phosphonium, sulfonium, or heterocyclyl;
A is alkyl, aryl, cycloalkyl, or heteroaryl; and
Y₂ is amido, amidino, oxygen, alkoxy, azo, carboxyl, alkenyl, ester, keto, phosphate, sulfoxide, sulfone, sulfonamido, heteroaryl, or heterocyclyl.

23. The compound of claim 14, wherein A is alkyl.

24. (canceled)

25. The polymeric nanoparticle of claim 14, wherein the transport moiety is represented by Formula V or a pharmaceutically acceptable salt thereof, 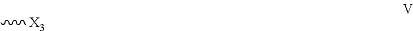
wherein
X₃ is amino, guanidino, hydrazino, diazonium, phosphonium, sulfonium, or heterocyclyl.

26. The polymeric nanoparticle of claim 14, wherein the transport moiety is represented by Formula VI or a pharmaceutically acceptable salt thereof, 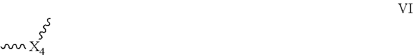
wherein
X₄ is amino, guanidino, hydrazino, phosphonium, sulfonium, or heterocyclyl.

27. (canceled)

28. (canceled)

29. The polymeric nanoparticle of claim 1, wherein the copolymer further comprises a copolymer of transport monomers, each coupled to a transport moiety, and cross-linking monomers, and the transport monomers are independently selected from 2-methacryloyloxyethyl phosphorylcholine (MPC), N-(3-aminopropyl) methacrylamide (APm), trimethyl(2-prop-2-enoyloxyethyl)azanium, methacrylatoethyl trimethyl ammonium, [2-(methacryloyloxy)ethyl]dimethyl-(3-sulfopropyl)ammonium hydroxide, quaternized dimethylaminoethyl methacrylate, carboxybetaine methacrylate, carboxybetaine acrylamide, vinyl pyridine, and poly(ethylene glycol) methyl ether acrylate monomers.

30-34. (canceled)

35. The polymeric nanoparticle of claim 1, wherein the cross-linking monomers are independently selected from N,N′-methylenebisacrylamide (BIS), bis[2-(methacryloyloxy)ethyl] phosphate (BMEP), glycerol dimethacrylate (GDMA), polylactide-based block copolymer bisacrylate and a bisacrylated peptide.

36-43. (canceled)

44. The polymeric nanoparticle of claim 29, wherein the copolymer further comprises stabilization monomers, and the stabilization monomers are polyethylene glycol monomers.

45. (canceled)

46. The polymeric nanoparticle of claim 7, wherein the copolymer further comprises agent monomers, each coupled to a bioactive agent, and the agent monomers are independently selected from methacrylate ester, methyacrylamide, N-acryloxysuccinimide (NAS), and N-(3-aminopropyl) methacrylamide (APm) monomers.

47. (canceled)

48. (canceled)

49. The polymeric nanoparticle of claim 46, wherein the agent monomers are coupled to the bioactive agent by a linker and the linker comprises either:
i) an alkyl chain, a heteroalkyl chain, and an alkenyl chain; or
ii) a plurality of linker monomers independently selected from N,N′-Methylenebis(acrylamide), and bis[2-(methacryloyloxy)ethyl] phosphate monomers.

50-52. (canceled)

53. The polymeric nanoparticle of claim 49, wherein the linker is degradable.

54-57. (canceled)

58. The polymeric nanoparticle of claim 1, wherein the bioactive agents are selected from small molecules, proteins, polynucleotides, and imaging agents.

59-70. (canceled)

71. The polymeric nanoparticle of claim 1, wherein the polymeric nanoparticle further comprises a plurality of targeting moieties and the targeting moieties are selected from targeting small molecules and targeting proteins.

72-80. (canceled)

81. The polymeric nanoparticle of claim 1, wherein the polymer further comprises at least one hydrophilic moiety and the hydrophilic moiety comprises at least one hydrogen bond acceptor.

82. (canceled)

83. The polymeric nanoparticle of claim 81, wherein the hydrophilic moiety is a neutral polymer or a zwitterionic polymer.

84. The polymeric nanoparticle of claim 81, wherein the hydrophilic moiety is poly(ethylene glycol), poly(vinylpyridine), poly(2-hydroxyethyl methacrylate), poly(oligoethylene glycol), poly(methacrylate), poly(acrylate), poly(amide), poly(peptoid), poly(oxazoline), poly(hydroxylethylacrylate), and poly(ethyl ethylene phosphate) or a poly-saccharide.

85. (canceled)

86. (canceled)

87. The polymeric nanoparticle of claim 83, wherein the zwitterionic polymer is a poly(phosphorylcholine), a poly(sulfobetaine), or a poly(carboxybetaine).

88-93. (canceled)

94. The polymeric nanoparticle of claim 1, wherein the nanoparticle is 5-20 nm in diameter.

95-143. (canceled)

144. A pharmaceutical composition comprising the polymeric nanoparticle of claim 1 and a pharmaceutically acceptable excipient.

145. A method of imaging the central nervous system of a subject in need thereof, comprising administering to the subject an effective amount of a polymeric nanoparticle of claim 1, wherein the polymeric nanoparticle comprises an imaging agent.

146. (canceled)

147. A method of treating a disease or disorder, comprising of administering to a subject in need of a treatment for said disease or disorder, a therapeutically effective amount of a polymeric nanoparticle of claim 1.

148-157. (canceled)

158. A method of making a polymeric nanoparticle of claim 1, comprising:
combining i) a plurality of transport monomers, each coupled to at least one transport moiety; ii) a plurality of cross-linking monomers; iii) a plurality of agent monomers, each coupled to at least one bioactive agent; and iv) a solvent; and
initiating copolymerization of the monomers with an initiator.

159-183. (canceled)
